SEQUENCE LISTING (27 sequences)

SEQ ID NO: 1 - protein, 353 aa, Artificial Sequence
  Description of Artificial Sequence Synthetic polypeptide; 2h-572-633-CT-L2
EVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, Artificial Sequence
  Description of Artificial Sequence Synthetic polypeptide; BMS2h-572-633
EVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSS

SEQ ID NO: 3 - protein, 232 aa, Artificial Sequence
  Description of Artificial Sequence Synthetic polypeptide; modified IgG1 Fc
EPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 261 aa, Homo sapiens
  MISC_FEATURE (1)..(261): CD40L
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 229 aa, Artificial Sequence
  Description of Artificial Sequence Synthetic polypeptide; modified IgG4 Fc
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 5 aa, Artificial Sequence
  Description of Artificial Sequence Synthetic polypeptide
GGGGS

SEQ ID NO: 7 - protein, 10 aa, Artificial Sequence
  Description of Artificial Sequence Synthetic polypeptide
GGGGSGGGGS

SEQ ID NO: 8 - protein, 15 aa, Artificial Sequence
  Description of Artificial Sequence Synthetic polypeptide
GGGGSGGGGSGGGGS

SEQ ID NO: 9 - protein, 20 aa, Artificial Sequence
  Description of Artificial Sequence Synthetic polypeptide
GGGGSGGGGSGGGGSGGGGS

SEQ ID NO: 10 - protein, 25 aa, Artificial Sequence
  Description of Artificial Sequence Synthetic polypeptide
GGGGSGGGGSGGGGSGGGGSGGGGS

SEQ ID NO: 11 - protein, 2 aa, Artificial Sequence
  Description of Artificial Sequence Synthetic polypeptide
AS

SEQ ID NO: 12 - protein, 3 aa, Artificial Sequence
  Description of Artificial Sequence Synthetic polypeptide
AST

SEQ ID NO: 13 - protein, 6 aa, Artificial Sequence
  Description of Artificial Sequence Synthetic polypeptide
TVAAPS

SEQ ID NO: 14 - protein, 3 aa, Artificial Sequence
  Description of Artificial Sequence Synthetic polypeptide
TVA

SEQ ID NO: 15 - protein, 7 aa, Artificial Sequence
  Description of Artificial Sequence Synthetic polypeptide
ASTSGPS

SEQ ID NO: 16 - protein, 10 aa, Artificial Sequence
  Description of Artificial Sequence Synthetic polypeptide
ASTHTCPPCP

SEQ ID NO: 17 - protein, 18 aa, Artificial Sequence
  Description of Artificial Sequence Synthetic polypeptide
ASTEPKSSDKTHTSPPSP

SEQ ID NO: 18 - protein, 10 aa, Artificial Sequence
  Description of Artificial Sequence Synthetic polypeptide
ASTHTSPPSP

SEQ ID NO: 19 - protein, 18 aa, Artificial Sequence
  Description of Artificial Sequence Synthetic polypeptide
ASTEPKSSDKTHTSPPSP

SEQ ID NO: 20 - protein, 10 aa, Artificial Sequence
  Description of Artificial Sequence Synthetic polypeptide
ASTHTSPPSP

SEQ ID NO: 21 - protein, 24 aa, Artificial Sequence
  Description of Artificial Sequence Synthetic polypeptide
MRAWIFFLLCLAGRALAEVQLLES

SEQ ID NO: 22 - protein, 370 aa, Artificial Sequence
  Description of Artificial Sequence Synthetic polypeptide
MRAWIFFLLCLAGRALAEVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 362 aa, Artificial Sequence
  Description of Artificial Sequence Synthetic polypeptide
MRAWIFFLLCLAGRALAEVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 370 aa, Artificial Sequence
  Description of Artificial Sequence Synthetic polypeptide
MRAWIFFLLCLAGRALAEVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 362 aa, Artificial Sequence
  Description of Artificial Sequence Synthetic polypeptide
MRAWIFFLLCLAGRALAEVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 108 aa, Artificial Sequence
  Description of Artificial Sequence Synthetic polypeptide; anti-CD28 dAb
DIQMTQSPSSLSASVGDRVTITCRASRPIWPFLEWYQQKPGKAPKLLIYFTSRLRHGVPSRFSGSGSGTCFTLTISSLQPEDFATYYCLQNVANPATFSQGTKVEIKR

SEQ ID NO: 27 - DNA, 354 bp, Artificial Sequence
  Description of Artificial Sequence Synthetic polynucleotide; BMS2h-572-633
gaggtgcagctgttggagtctgggggaggcttggtacagcctggggggtccctgcgtctctcctgtgcagcctccggattcacctttaactgggagctgatgggctgggcccggcaggctccagggaagggtctagagtgggtctcaggtattgagggtccaggtgatgttacatactacgcagactccgtgaagggccggttcaccatctcccgcgacaattccaagaacacgctgtatctgcaaatgaacagcctgcgtgccgaggataccgcggtatattactgtgtgaaagtggggaaggacgccaagtcggactaccggggtcagggaaccctggtcaccgtatcgagc

(19) United States
(12) Patent Application Publication
(10) Pub. No.: US 2017/0051059 A1
(43) Pub. Date: Feb. 23, 2017

(54) METHODS OF TREATING TRANSPLANT REJECTION USING A DOMAIN ANTIBODY DIRECTED AGAINST CD40

(71) Applicant: Bristol-Myers Squibb Company, Princeton, NJ (US)

(72) Inventors: Anish SURI, Yardley, PA (US); Steven G. NADLER, Princeton, NJ (US); Christian P. LARSEN, Atlanta, GA (US); Andrew Briane ADAMS, Lilburn, GA (US)

(73) Assignees: Bristol-Myers Squibb Company, Princeton, NJ (US); Bristol-Myers Squibb Company, Princeton, NJ (US)

(21) Appl. No.: 15/122,455

(22) PCT Filed: Mar. 19, 2015
(86) PCT No.: PCT/US15/21551

§ 371 Date: Aug. 30, 2016

Related U.S. Application Data
(60) Provisional application No. 61/955,588, filed on Mar. 19, 2014.

(51) Int. Cl. C07K 16/28; A61K 39/395; A61K 38/17; C07K 14/705

(52) U.S. Cl. CPC C07K 16/2818; C07K 16/2875; C07K 14/70521; A61K 39/3955; A61K 38/1774; C07K 2317/40; C07K 2317/569; A61K 2039/545

(57) ABSTRACT

Methods of treating renal transplant rejection using anti-CD40L domain antibodies are provided. The anti-CD40L dAbs are less likely to cause platelet aggregation and thus cause thromboembolism. Appropriate anti-CD40L dAbs doses and administration regimens are also provided. Combination treatments for transplant rejection, particularly renal transplant rejection, using anti-CD40L dAbs, a CTLA4 mutant molecule (e.g., belatacept) and/or anti-CD28 optionally with conventional immunosuppressive renal transplant therapy are provided.

### REFERENCE TO RELATED APPLICATIONS

This application claims benefit to U.S. Provisional Application No. 61/955,588 filed Mar. 19, 2014, which is hereby incorporated in its entirety for all purposes.

### TECHNICAL FIELD

Methods of treating transplant rejection, particularly renal transplant rejection, using anti-CD40L dAbs are provided. Appropriate anti-CD40L dAbs doses and administration regimens are also provided. In addition, combination treatments for transplant rejection, particularly renal transplant rejection, using anti-CD40L dAbs and a CTLA4 antibody are provided.

### REFERENCE TO A SEQUENCE LISTING

The instant application contains a Sequence Listing, which has been submitted electronically in ASCII format and is hereby incorporated by reference in its entirety. Said ASCII copy, created on Mar. 19, 2015, is named 200896_522603_SL.txt and is 30,328 bytes in size.

### BACKGROUND

Transplantation is a therapy for end-stage organ failure, with more than 25,000 solid organ transplants performed annually in the US. Since the introduction of calcineurin inhibitor therapy almost 30 years ago, the incidence of early graft failure due to acute rejection has been dramatically reduced. However, long-term graft survival remains less than ideal. Immune and non-immune mediated chronic graft injury can result in progressive loss of allograft function. Chronic graft injury can be attributed, in part, to the non-immune side effects associated with current immunosuppressive therapy, in particular calcineurin inhibitors. In recent years, many of the pathways involved in T cell activation and function have been elucidated, including pathways involving cell surface proteins involved in T cell co-stimulation. In an effort to more specifically inhibit T cell mediated rejection and avoid the side effects associated with current immunosuppressive agents, novel biologic agents directed against pathways involved in T cell activation have been developed.

Among these agents are anti-CD40L antibodies. The role of CD40-CD40L interactions in immune and inflammatory responses has made them a promising target for treatment of pathological immuno-inflammatory processes. Blockade of CD40-CD40L interactions by means of specific CD40L monoclonal antibodies (mAbs) successfully prevents allograft rejection in primates and treats autoimmune diseases and atherosclerosis in animal models. Montgomery et al., Transplantation 74: 1365-1369 (2002).

In humans, two different anti-CD40L monoclonal antibody (mAb) clones have been used in clinical trials for treatment of different autoimmune diseases. Maribel et al., Mol. Immunol. 45: 937-44 (2008). Monoclonal antibodies, however, can display unusually high incidence of thromboembolic (TE) complications, such as atherothrombotic central nervous system events, myocardial infarction, pulmonary embolism, and deep vein thrombosis. For example, the usefulness of the anti-CD40L mAb clone hu5c8 (anti-CD40L mAb, Biogen) is limited by an unusually high incidence of TE complications. TE complications induced by these antibodies is thought to result from the formation of higher-order immune complexes (IC) of the mAbs with membrane-bound CD40L on platelets, or sCD40L shed from platelets, that can ligate and thereby aggregate neighboring platelets via their FcgRIIa receptors, resulting in thrombi formation. The risk of thromboembolism has led to a halt in all ongoing clinical trials. Boumpas et al., Arthritis & Rheumatism 48: 719-727 (2003).

Thus, the present invention fulfills a need in the art by providing methods of treating transplant rejection using a domain antibody that targets CD40L, but does not cause for example, thromboembolisms (TE).

### SUMMARY

Methods of treating transplant rejection, in particular renal transplant rejection that do not cause or have a lower risk of thromboembolism (TE) are still needed for clinical use. Such methods can include dosage regimens and administration routes for anti-CD40L antibody antagonists that are less likely to cause platelet aggregation and thus less likely to cause thromboembolism.

A method of treating renal transplant rejection can comprise administering a therapeutically effective amount of BMS2h-572-633-CT-L2 (SEQ ID NO: 1) to a patient in need thereof.

The transplant rejection can be an acute transplant rejectionor a chronic transplant rejection.

The method of treating renal transplant rejection can comprise administering a BMS2h-572-633-CT-L2 (SEQ ID NO: 1) dose from about 2 to about 30 mg/kg patient weight. The method of treating renal transplant rejection can also comprise administering a BMS2h-572-633-CT-L2 (SEQ ID NO: 1) dose at about 20 to about 30 mg/kg patient weight. The method of treating renal transplant rejection can comprise administering a BMS2h-572-633-CT-L2 (SEQ ID NO: 1) dose about 20 mg/kg patient weight.

BMS2h-572-633-CT-L2 (SEQ ID NO: 1) can be administered with an immunosuppressive/immunomodulatory and/or anti-inflammatory agent. The immunosuppressive, immunomodulatory and/or anti-inflammatory agent can be a CTLA4 mutant molecule. The CTLA4 mutant molecule can be L104EA29Y-Ig (Belatacept). L104EA29Y-Ig (Belatacept) can be administered at a dose from about 10 to about 20 mg/kg patient weight. Alternatively, Belatacept can be administered at a dose of about 20 mg/kg patient weight.

BMS2h-572-633-CT-L2 (SEQ ID NO: 1) can be administered on a weekly basis during the duration of the treatment regimen. The immunosuppressive, immunomodulatory and/or anti-inflammatory agent can be administered together with BMS2h-572-633-CT-L2 (SEQ ID NO: 1) on a weekly basis during the duration of the treatment regimen. The duration of the treatment regimen can be about 70 days.

BMS2h-572-633-CT-L2 (SEQ ID NO: 1) can be administered intravenously. The immunosuppressive, immunomodulatory and/or anti-inflammatory agent can be administered intravenously.

BMS2h-572-633-CT-L2 (SEQ ID NO: 1) can be administered alone, or in combination with a conventional therapy for the treatment of renal transplant rejection. An exemplary conventional therapy for use along with BMS2h-572-633-CT-L2 is a combination of an anti-IL-2R antibody, solumedrol, and mycophenolate mofetil (MMF). The conventional therapy can then be tapered off over time as indicated by the patient's progress.

The immunosuppressive/immunomodulatory and/or anti-inflammatory agent can be an anti-CD28 dAb. The anti-CD28 dAb can comprise SEQ ID NO: 26, which can optionally be pegylated. One example of an anti-CD28 dAb is BMS-931699 (otherwise referred to as 1h-239-891 (D70C) P30L-PEG or 239-891-D70C P30L PEG), which is a PEGylated anti-CD28 dAb. The PEG moiety can be a 40 kDa branched polyethylene glycol. The anti-CD28 dAb can be administered at a dose of about 1 mg/kg to about 10 mg/kg patient weight in combination with the BMS2h-572-633-CT-L2. One exemplary dosage is about 3 mg/kg of the anti-CD28 dAb and can be administered at weekly intervals.

The methods described herein can be alternatively considers as a use of BMS2h-572-633-CT-L2 (SEQ ID NO: 1) for the preparation of a medicament for treating renal transplant rejection in a patient in therapeutic need thereof. The use of BMS2h-572-633-CT-L2 (SEQ ID NO: 1) can be applied to any of the methods and combinations described above and infra.

### BRIEF DESCRIPTION OF THE FIGURES

FIG. 1A depicts in ribbon format the domain antibody that comprises a V_{H} variable domain BMS2h-572-633 fused to the modified Fc tail from Abatacept IgG1.

FIG. 1B shows the amino acid sequence (SEQ ID NO: 1) of BMS2h-572-633-CT-L2, comprising the variable domain BMS2h-572-633 (SEQ ID NO: 2). The Fc fusion protein is a dimer of molecular weight 77,984 Daltons, with each polypeptide chain consisting of 354 amino acids. The variable domain is fused by a linker to a mutated Fc construct of human IgG1, wherein three cysteine residues are substituted with serine, and one proline is substituted with a serine residue (SEQ ID NO: 3).

FIG. 2 provides the N-terminal amino acid sequences (SEQ ID NOS: 1356-1361, respectively, from top to bottom of various Fc domains linked to linkers. Linker regions are shown in boxes.

FIG. 3 shows examples of various Fc-formatted domain antibodies (SEQ ID NOS 1362-1365, respectively, in order of appearance). Linker regions are indicated by boxes.

FIG. 4 depicts SPR sensorgram data for the binding of 12.5-0.39 nM BMS-986004 (2:1 dilution series) to biot-IZ-hCD40L captured on a streptavidin SPR sensor chip at 25° C. Colored lines show the double-referenced sensorgram data, and black lines show the 1:1 Langmuir fit to the data, with an avidity-influenced apparent Kd value of 0.11 nM.

FIG. 5 shows ITC data for titrations of 19 μM IZ-hCD40L into 2 μM BMS-986004 (black) or 18 μM BMS-986004 into 2 μM IZ-hCD40L (blue). The molar ratio (apparent stoichiometry) is defined per mole of IZ-hCD40L trimer and per mole of bivalent BMS-986004 Fc-dimer. Molar ratio values obtained as the equivalence points on the abscissa suggest more than one mole of BMS-986004 can bind per mole of IZ-hCD40L trimer; however, an exact structural model for the complex cannot be determined from the ITC data alone. Squares represent the integrated heat of binding data and solid lines represent the best fit to a “2 sets of sites model.”

FIG. 6 shows in vivo efficacy of mouse CD40L surrogate dAb-Fc (KLH-induced antibody response) 2 panels).

FIG. 7 demonstrates that mouse dAb BMS-2m-126-24-Fc and antibody MR-1 inhibit TNBS-induced colitis in mice (4 panels).

FIG. 8 shows that BMS-2m-126-24-Fc and CTLA4-Ig work synergistically to prolong the survival of cardiac allografts.

FIG. 9A shows plasma concentration vs. time profile of BMS-986004 after IV dosing of 11 mg/kg in monkeys.

FIG. 9B demonstrates plasma concentration vs. time profiles of BMS-986003 after IV dosing of 2 mg/kg in monkeys.

FIG. 10 presents plasma concentrations vs. time profiles of BMS-986003 (after SC dosing at 0.2, 2.0 and 20 mg/kg in monkeys) and of 5c8 IgG1 (after IV dosing at 20 mg/kg in monkeys).

FIG. 11 shows plasma concentrations vs. time profiles of BMS-2m-126-24-CT after 1 mg/kg IV and SC dosing, and 10 mg/kg SC dosing in mice.

FIG. 12 demonstrates PK/PD modeling of BMS-986003 and 5c8-IgG1 plasma exposures and anti-KLH antibody response (IgG Titers)(4 panels).

FIG. 13 shows PK/PD modeling of BMS-986004 plasma exposures (left) and ex vivo RO on peripheral blood mononuclear cells (PBMC)(right).

FIG. 14 demonstrates that IV.3 blocks 5c8/sCD40L IC-mediated activation of platelets in human blood.

FIG. 15 shows the effect of Fe variants on platelet activation in human blood.

FIG. 16 demonstrates activation of platelets with 5c8-CT/sCD40L IC in blood from human donors genotyped for FcgRIIa polymorphism.

FIG. 17 diagrams platelet activation by various antibodies in blood from human donors.

FIG. 18 shows levels of platelet activation by various antibodies, including BMS-986003, in hFcgRIIa-expressing transgenic mice.

FIG. 19 presents serum creatinine (mg/dL) curves for renally transplanted monkeys treated with high doses (20 mg/kg intravenously) of BMS2h-572-633-CT-L2 (SEQ ID NO: 1).

FIG. 20 presents serum creatinine (mg/dL) curves for renally transplanted monkeys treated with intermediate doses (10 mg/kg intravenously) of BMS2h-572-633-CT-L2 (SEQ ID NO: 1).

FIG. 21 presents serum creatinine (mg/dL) curves for renally transplanted monkeys treated with low doses (2 mg/kg intravenously) of BMS2h-572-633-CT-L2 (SEQ ID NO: 1).

FIG. 22 presents serum creatinine (mg/dL) curves for renally transplanted monkeys treated with high doses (30 mg/kg intravenously) of BMS2h-572-633-CT-L2 (SEQ ID NO: 1).

FIG. 23 presents serum creatinine (mg/dL) curves for renally transplanted monkeys treated with high doses (20 mg/kg intravenously) of BMS2h-572-633-CT-L2 (SEQ ID NO: 1).

FIG. 24 presents flow cytometry diagrams showing leukocyte composition (immunophenotype) in peripheral blood and other peripheral blood cellular markers consistent with immune activation (CD3+, CD4+, CD8+ T cells) in renally transplanted monkeys treated with 20 mg/kg BMS2h-572-633-CT-L2 (SEQ ID NO: 1).

FIG. 25 presents flow panels for the cytometry diagrams of FIG. 24.

FIG. 26 shows CD4+/CD8+ naïve T cell compositions in the peripheral blood of renally transplanted rhesus monkeys treated with 20 mg/kg of BMS2h-572-633-CT-L2 (SEQ ID NO: 1) intravenously.

FIG. 27 shows CD4+/CD8+ memory T cell compositions in the peripheral blood of renally transplanted rhesus monkeys treated with 20 mg/kg of BMS2h-572-633-CT-L2 (SEQ ID NO: 1) intravenously.

FIG. 28 shows CD4+/CD8+ memory T cell compositions in the peripheral blood of renally transplanted rhesus monkeys treated with 20 mg/kg of BMS2h-572-633-CT-L2 (SEQ ID NO: 1) intravenously.

FIG. 29 shows CD4+/CD8+ naïve T cell compositions in the peripheral blood of renally transplanted rhesus monkeys treated with 20 mg/kg of BMS2h-572-633-CT-L2 (SEQ ID NO: 1) intravenously and 20 mg/kg of Belatacept.

FIG. 30 shows CD4+/CD8+ memory T cell compositions as in the peripheral blood of renally transplanted rhesus monkeys treated with 20 mg/kg of BMS2h-572-633-CT-L2 (SEQ ID NO: 1) intravenously and 20 mg/kg of Belatacept.

FIG. 31 shows CD4+/CD8+ memory cell compositions as in the peripheral blood of renally transplanted rhesus monkeys treated with 20 mg/kg of BMS2h-572-633-CT-L2 (SEQ ID NO: 1) intravenously and 20 mg/kg of Belatacept.

FIG. 32 demonstrates cytomegalovirus (CMV) viral reactivation rates (copies/mL) in Rhesus monkey's treated with BMS2h-572-633-CT-L2 (SEQ ID NO: 1) at 20 mg/kg.

### DETAILED DESCRIPTION

Methods of treating renal transplant rejection using antibody polypeptides that specifically bind to human CD40L are provided. The antibody polypeptides are less likely to cause platelet aggregation and are thus less likely to cause thromboembolism.

As used herein, “specific binding” refers to the binding of an antigen by an antibody polypeptide with a dissociation constant (K_{d}) of about 1 μM or lower as measured, for example, by surface plasmon resonance (SPR). Suitable assay systems include the BIAcore™ surface plasmon resonance system and BIAcore™ kinetic evaluation software (e.g., version 2.1). The affinity or K_{d} for a specific binding interaction may be about 1 μM or lower, about 500 nM or lower or about 300 nM or lower.

The term “about” will be understood by persons of ordinary skill in the art and will vary to some extent on the context in which it is used. Generally, about encompasses a range of values that are plus/minus 10% of a referenced value.

In accordance with this detailed description, the following abbreviations and definitions apply. It must be noted that as used herein, the singular forms “a”, “an”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

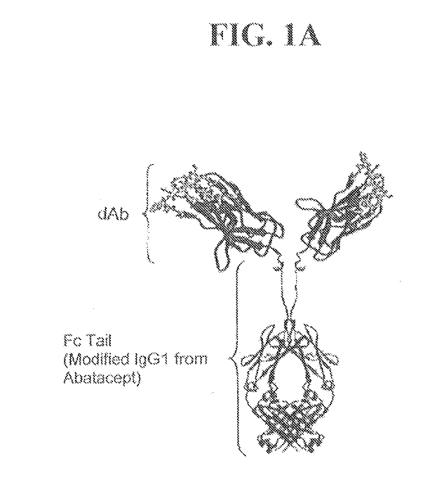

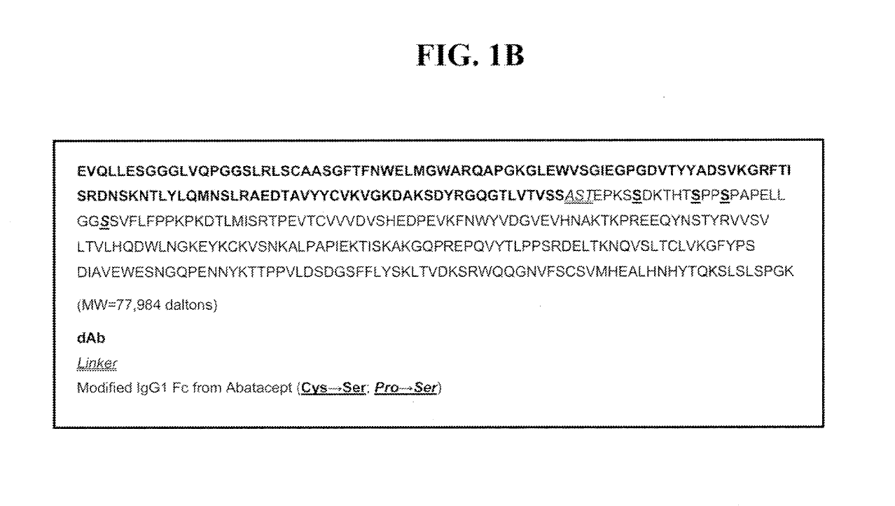

As used herein, “BMS-986004” refers to a dimeric fusion polypeptide, composed of two molecules of an antibody polypeptide having a modified Fc fragment of IgG1 linked to the C-terminus of the dAb BMS2h-572-633, via an intervening linker sequence having the amino acid sequence, AST. BMS2h-572-633 dAb has the amino acid sequence of SEQ ID NO: 2. An exemplary coding sequence for BMS2h-572-633 dAb is SEQ ID NO: 27. The modified Fc fragment has the amino acid sequence of SEQ ID NO: 3. See FIGS. 1A and 1B. Other names for BMS-986004 used herein include BMS2h-572-633-CT-L2, 2h-572-633-CT-L2, BMS2h-572-633-CT-long, and 2h-572-633-CT-long.

It is understood that any and all whole or partial integers between the ranges set forth here are included herein.

1. CD40L and CD40L Activities

Antibody polypeptides are provided that bind human CD40L. CD40L is also known as CD154, gp39, TNF-related activation protein (TRAP), 5c8 antigen, or T-BAM. Relevant structural information for human CD40L can be found, for example, at UniProt Accession Number P29965. “Human CD40L” refers to the CD40L comprising the following amino acid sequence:

```
(SEQ ID NO: 3)
                10 20 30 40 50 60
MIETYNQTSP RSAATGLPIS MKIFMYLLTV FLITQMIGSA LFAVYLHRRL DKIEDERNLH
                70 80 90 100 110 120
EDFVFMKTIQ RCNTGERSLS LLNCEEIKSQ FEGFVKDIML NKEETKKENS FEMQKGDQNP
              130 140 150 160 170 180
QIAAHVISEA SSKTTSVLQW AEKGYYTMSN NLVTLENGKQ LTVKRQGLYY IYAQVTFCSN
              190 200 210 220 230 240
REASSQAPFI ASLCLKSPGR FERILLRAAN THSSAKPCGQ QSIHLGGVFE LQPGASVFVN
              250 260
VTDPSQVSHG TGFTSFGLLK L
```

CD40L has also been sequenced in Sus scrofa, Mus musculus, Canis familiaris, Bos ffini, Macaca mulatta, Aotus tivirgalus, Callithrix jacchus, Cercocebus torquatus atys, Macaca nemestrina, Rattus norvegicus, Gallus gallus, Felis calus, and Sus scrofa.

Binding of the present antibody polypeptides to CD40L antagonizes CD40L activity. “CD40L activities” include, but are not limited to, co-stimulation and activation an APC in association with T cell receptor stimulation by MHC molecules on the APC, secretion of all immunoglobulin isotypes in the presence of cytokines, stimulation of B cell proliferation, cytokine production, antibody class switching and affinity maturation. For example, patients with X-linked hyper-IgM syndrome express functional CD40 on their B cells, but their activated T cells have a defective CD40L protein, resulting in its inability to activate B cells and induce immunoglobulin isotype switching. Aruffo et al., Cell 72: 291-300 (1993).

CD40L activities can be mediated by interaction with other molecules. “CD40 activities” include the functional interaction between CD40L and the following molecules: CD40 (CD40L receptor), α5β1 integrin, and αIIbβ3. For example, CD40L binds its receptor, CD40, which is expressed on a variety of APCs, such as B cells, macrophages, and dendritic cells, as well as on stromal cells, vascular endothelial cells, and platelets.

As used herein, the terms “activate,” “activates,” and “activated” refer to an increase in a given measurable CD40L activity by at least 10% relative to a reference, for example, at least 10%, 25%, 50%, 75%, or even 100%, or more. A CD40L activity is “antagonized” if the activity is reduced by at least 10%, and in an exemplary embodiment, at least 20%, 30%, 40%, 50%, 60%, 70%, 80%, 90%, 95%, 97%, or even 100% (i.e., no detectable activity), relative to the absence of the antagonist. For example, an antibody polypeptide may antagonize some or all CD40L activity. The antibody polypeptide may not activate B cell proliferation. The antibody polypeptide may not activate cytokine secretion by T cells or dendritic cells (DCs), where the cytokine is at least one cytokine selected from the group consisting of IL-2, IL-6, IL-10, IL-12, IL-13, IL-17, IL-23, TNF-α, and IFN-γ.

2. Antibody Polypeptides

The antibody polypeptides comprise a variable domain. The antibody polypeptides can be in the form of a dAb that contains a single variable domain. Antibody polypeptides may be full-length anti-CD40L immunoglobulin molecules comprising two heavy (H) chains and two light (L) chains interconnected by disulfide bonds. The amino terminal portion of each chain includes a variable domain (V_{L} or V_{H}) of about 100-120 amino acids. The complementarity determining regions (CDRs) contained therein are primarily responsible for antigen recognition, although framework residues can play a role in epitope binding. The carboxy-terminal “half” of each heavy chain defines a constant region (Fc) primarily responsible for effector function.

A “domain antibody” (dAb) comprises a single variable (V_{L} or V_{H}) domain that is capable of specifically and monovalently binding an antigen, such as CD40L. For example, a dAb may have a V_{HH} structure, characteristic of a camelid dAb. A “V_{H} domain” as used herein is meant to include a V_{HH} structure. The V_{H} domains (including all features and combination of features presented as embodiments herein) are other than V_{HH} domains. dAbs may form homo- or heterodimers in solution. While not limited by any particular theory, it is believed that the dAbs disclosed herein do not cause platelet aggregation, because the antibodies containing mutated Fc constructs do not bind FcγRIIa (also known as CD32a) on the platelet surface and do not activate platelets.

As used herein, the term “variable domain” refers to immunoglobulin variable domains defined by Kabat et al., Sequences of Immunological Interest, 5^{th} ed., U.S. Dept. Health & Human Services, Washington, D.C. (1991). The numbering and positioning of CDR amino acid residues within the variable domains is in accordance with the well-known Kabat numbering convention.

Antibody polypeptides also may be “fragments” comprising a portion of the full-length anti-CD40L immunoglobulin molecule that comprises a variable domain that specifically binds CD40L. Thus, the term “antibody polypeptides” includes an antigen-binding heavy chain, light chain, heavy chain-light chain dimer, Fab fragment, F(ab′)₂ fragment, Fv fragment, single chain Fv (scFv), and dAb, for example. The term “antibody polypeptides” thus includes polypeptides made by recombinant engineering and expression, as well as monoclonal antibodies produced by natural recombination and secretion by hybridoma cell clones.

Light chains are classified as kappa (κ) or lambda (λ), and are characterized by a particular constant region, C_{L}, as known in the art. Heavy chains are classified as γ, μ, α, δ, or ε, and define the isotype of an antibody as IgG, IgM, IgA, IgD, or IgE, respectively. The heavy chain constant region is comprised of three domains (CH1, CH2, and CH3) for IgG, IgD, and IgA; and four domains (CH1, CH2, CH3, and CH4) for IgM and IgE. Anti-CD40L antibodies may have a heavy chain constant region selected from any of the immunoglobulin classes (IgA, IgD, IgG, IgM, and IgE).

Each light chain variable domain (V_{L}) and heavy chain variable domain (V_{H}) is composed of three CDRs and four framework regions (FRs), arranged from amino-terminus to carboxy-terminus in the following order: FR1, CDR1, FR2, CDR2, FR3, CDR3, and FR4. The three CDRs of the light chain are referred to as “LCDR1, LCDR2, and LCDR3” and the three CDRs of the heavy chain are referred to as “HCDR1, HCDR2, and HCDR3.”

As used herein, the term “Fc domain” refers to the constant region antibody sequences comprising CH2 and CH3 constant domains as delimited according to Kabat et al., Sequences of Immunological Interest, 5^{th} ed., U.S. Dept. Health & Human Services, Washington, D.C. (1991). The Fc region may be derived from a human IgG. The Fc domain may be derived from a human IgG1 or a human IgG4 Fc region, for example. An exemplary modified human IgG Fc domain is:

(SEQ ID NO: 3)
EPKSSDKTHTSPPSPAPELLGGSSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

SEQ ID NO: 3 is derived from human IgG1 Fc, and comprises Ser at positions 5, 11 and 14 instead of Cys, and position 23 comprises Ser instead of Pro. The cysteine-to-serine point mutations are made to eliminate the disulfides in the Fc hinge. Another exemplary Fc region is SEQ ID NO: 5, which is derived from human IgG4 Fc, has the amino acid sequence:

(SEQ ID NO: 5)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

SEQ ID NO: 5 is derived from human IgG4 Fc and comprises a modification of position 10 to comprise Pro.

A variable domain may be fused to an Fc domain. When a variable domain is fused to an Fc domain, the carboxyl terminus of the variable domain (either a V_{L} or V_{H} domain, including dAbs) may be linked or fused to the amino terminus of the Fc CH2 domain. Alternatively, the carboxyl terminus of the variable domain may be linked or fused to the amino terminus of a CH1 domain, which itself is fused to the Fc CH2 domain. The protein may comprise the hinge region between the CH1 and CH2 domains in whole or in part.

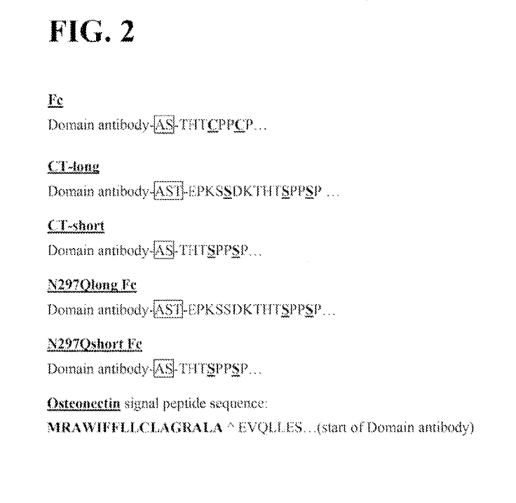

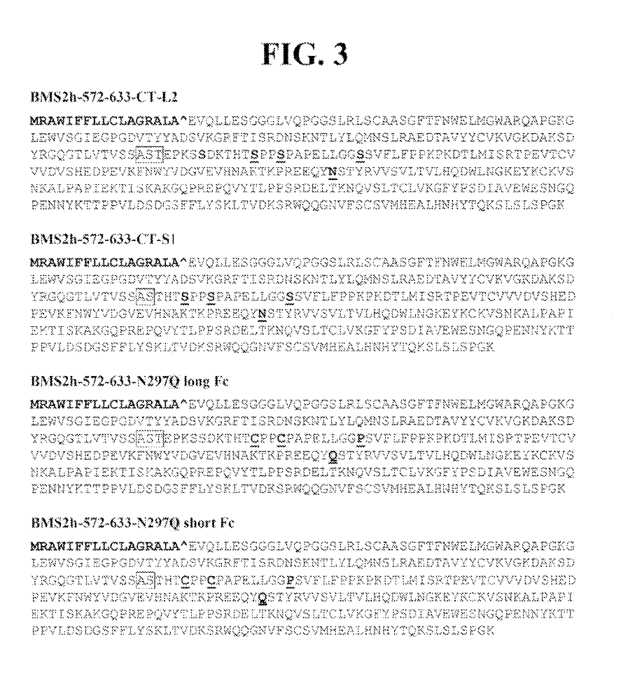

Examples of various Fc-formatted domain antibodies and their potency are provided in TABLE 4. FIG. 2 provides the N-terminal sequences of various Fc domains provided herein, linked to a linker region. Linker regions are shown in boxes. As used in TABLE 2, “Fc” indicates that the dAb is fused to a human IgG1 short Fc. “CT Long Fc,” also called CT-L2, CT long, and CT, has the amino acid sequence SEQ ID No: 3. “CT Short,” also called CT-S1, is shorter than CT Long by 7 amino acids at the N-terminus. “N297Q Long Fc,” also referred to as N297Q-L4, is the Fc domain of human IgG1 with a N297Q mutation made to eliminate the N-linked carbohydrate in the Fc. “N297Q Short Fc,” also called N297Q-S3, is short than N297Q Long Fc by 7 amino acids at the N-terminus, and is a human IgG1 with a N297Q point mutation made to eliminate the N-linked carbohydrate in the Fc domain. “CT-Fc SP5” is the CT Long Fc, where SP5 refers to the octeonectin signal peptide used for secretion from the mammalian expression host. Cleavage site is indicated by “̂” FIG. 3 further provides examples of various Fc domain formats.

Antibody polypeptides of a fusion antibody polypeptide may be linked by an “amino acid linker” or “linker.” For example, a dAb may be fused to the N-terminus of an amino acid linker, and an Fc domain may be fused to the C-terminus of the linker. Although amino acid linkers can be any length and consist of any combination of amino acids, the linker length may be relatively short (e.g., five or fewer amino acids) to reduce interactions between the linked domains. The amino acid composition of the linker also may be adjusted to reduce the number of amino acids with bulky side chains or amino acids likely to introduce secondary structure. Suitable amino acid linkers include, but are not limited to, those up to 3, 4, 5, 6, 7, 10, 15, 20, or 25 amino acids in length. Representative amino acid linker sequences include GGGGS (SEQ ID NO: 6), and linker comprising 2, 3, 4, or 5 copies of GGGGS (SEQ ID NOs: 7-10, respectively). The list below suitable linker sequences for use in the present disclosure.

SEQ ID NO: 6
GGGGS
SEQ ID NO: 7
(GGGGS)₂
SEQ ID NO: 8
(GGGGS)₃
SEQ ID NO: 9
(GGGGS)₄
SEQ ID NO: 10
(GGGGS)₅
SEQ ID NO: 11
AS
SEQ ID NO: 12
AST
SEQ ID NO: 13
TVAAPS
SEQ ID NO: 14
TVA
SEQ ID NO: 15
ASTSGPS

The first variable domain comprises the amino acid sequence of BMS2h-572-633 (SEQ ID NO: 2) is fused to a human Fc domain. See FIGS. 1A and 1B. The linker can be selected from any of the linkers list in the above Table. For example, the linker can comprise or be AS (SEQ ID NO: 11). Additionally, the method using the antibody polypeptide can comprise a variable domain wherein the amino acid sequence of the variable domain comprises BMS2h-572-633 (SEQ ID NO: 2), a linker comprising AST (SEQ ID NO: 12), and a human Fc domain selected from SEQ ID NO: 3. Another disclosed method, the antibody polypeptide comprises a variable domain wherein the amino acid sequence of the variable domain comprises BMS2h-572-633 (SEQ ID NO: 2), a linker comprising AS (SEQ ID NO: 11), and a human Fc domain comprising the amino acid sequence of SEQ ID NO: 5.

The term “human,” when applied to antibody polypeptides, means that the antibody polypeptide has a sequence, e.g., framework regions and/or CH domains, derived from a human immunoglobulin. A sequence is “derived from” a human immunoglobulin coding sequence when the sequence is either: (a) isolated from a human individual or from a cell or cell line from a human individual; (b) isolated from a library of cloned human antibody gene sequences or of human antibody variable domain sequences; or (c) diversified by mutation and selection from one or more of the polypeptides above. An “isolated” compound as used herein means that the compound is removed from at least one component with which the compound is naturally associated with in nature.

Antibody polypeptides can be administered to human patients while largely avoiding the anti-antibody immune response often provoked by the administration of antibodies from other species, e.g., mouse. For example, murine antibodies can be “humanized” by grafting murine CDRs onto a human variable domain FR, according to procedures well known in the art. Human antibodies as disclosed herein, however, can be produced without the need for genetic manipulation of a murine antibody sequence.

Variable domains may comprise one or more FR with the same amino acid sequence as a corresponding framework region encoded by a human germline antibody gene segment. For example, a domain antibody may comprise the V_{H} germline gene segments DP47, DP45, or DP38, the V_{κ} germline gene segment DPK9, the J_{H} segment JH4b, or the J_{κ} segment J_{κ}1.

Changes may be made to antibody polypeptide sequences while retaining the ability to bind CD40L specifically. Specifically, the antibody polypeptides (e.g., a dAb) may comprise a variant variable domain that retains the function of specifically binding CD40L as the dAb BMS2h-572-633. The variant variable domain may compete with BMS2h-572-633 for specific binding to CD40L.

An antibody polypeptide may be formatted to increase its in vivo half-life by PEGylation. The PEG is covalently linked. Alternatively, the PEG is linked to the antibody polypeptide at a cysteine or lysine residue. The PEG-linked antibody polypeptide can have a hydrodynamic size of at least 24 kD. Generally, the total PEG size is from 20 to 60 kD, inclusive. Generally, the PEG-linked domain antibody has a hydrodynamic size of at least 200 kD.

PEGylation can be achieved using several PEG attachment moieties including, but not limited to N-hydroxylsuccinimide active ester, succinimidyl propionate, maleimide, vinyl sulfone, or thiol. A PEG polymer can be linked to an antibody polypeptide at either a predetermined position, or can be randomly linked to the domain antibody molecule. PEGylation can also be mediated through a peptide linker attached to a domain antibody. That is, the PEG moiety can be attached to a peptide linker fused to an antibody polypeptide, where the linker provides the site (e.g., a free cysteine or lysine) for PEG attachment. Methods of PEGylating antibodies are known in the art, as disclosed in Chapman, et al., “PEGylated antibodies and antibody fragments for improved therapy: a review,” Adv. Drug Deliv. Rev. 54(4): 531-45 (2002), for example.

3. Pharmaceutical Compositions and Methods of Treatment

The method comprises administering an antibody polypeptide to a patient. The antibody polypeptide may be formulated as a pharmaceutical composition. A pharmaceutical composition comprises a therapeutically-effective amount of one or more antibody polypeptides and optionally a pharmaceutically acceptable carrier. Pharmaceutically acceptable carriers include, for example, water, saline, phosphate buffered saline, dextrose, glycerol, ethanol and the like, as well as combinations thereof. Pharmaceutically acceptable carriers can further comprise minor amounts of auxiliary substances, such as wetting or emulsifying agents, preservatives, or buffers that enhance the shelf-life or effectiveness of the fusion protein. The compositions can be formulated to provide quick, sustained, or delayed release of the active ingredient(s) after administration. Suitable pharmaceutical compositions and processes for preparing them are well known in the art. See, e.g., Remington, THE SCIENCE AND PRACTICE OF PHARMACY, A. Gennaro, et al., eds., 21^{st} ed., Mack Publishing Co. (2005).

The pharmaceutical composition further may comprise an immuno-suppressive/immunomodulatory and/or anti-inflammatory agent. A method of treating transplant rejection in a patient in need of such treatment may comprise administering to the patient a therapeutically effective amount of the pharmaceutical composition. The transplant can be a renal transplant. Antagonizing CD40L-mediated T cell activation could inhibit undesired T cell responses occurring during transplant rejection. Inhibiting CD40L-mediated T cell activation could moderate the progression and/or severity of transplant rejection.

As used herein, a “patient” means an animal, e.g. mammal, including humans. The patient may be diagnosed with an immune disease. “Treatment” or “treat” or “treating” refers to the process involving alleviating the progression or severity of a symptom, disorder, condition, or disease.

The pharmaceutical composition may be administered alone or in combination therapy (i.e., simultaneously, sequentially or co-formulated with) with an immunosuppressive/immunomodulatory and/or anti-inflammatory agent. Different immune diseases can require use of specific auxiliary compounds useful for treating immune diseases, which can be determined on a patient-to-patient basis.

For example, the disclosed pharmaceutical composition may be co-administered, concomitantly (simultaneously or co-formulated with) or sequentially, with a cytotoxic T-lymphocyte antigen 4 (CTLA4) mutant molecule, such as L104EA29Y-Ig (belatacept). CTLA4 binds to CD80 (B7-1) and CD86 (B7-2) with higher avidity than CD28, and it is transiently expressed on T cells following their activation, where it interrupts the interaction between CD28 and CD80/86. Oosterwegel et al., Curr. Opin. Immunol. 11: 294-300 (1999). This creates a negative feedback signal for T cell activation.

CTLA4 mutant molecules, including L104EA29Y-Ig, have increased binding avidity to CD80/86 compared to wild-type CTLA4. Intervention of the CD28-CD80/86 pathway by L104EA29Y-Ig has been successfully pursued, for example, to treat graft-related diseases in non-human primate transplant models, alone or in combination with other immunosuppressive agents. Larsen et al., Amer. J. Transplant. 5: 443 (2005). U.S. Patent Application number 2010/0166774 describes the structure of L104EA29Y-Ig, methods of producing L104EA29Y-Ig, and a formulation comprising a CTLA4 molecule; and the application is herein incorporated by reference. U.S. Pat. Nos. 7,094,874 and 7,482,327 further disclose administration (including co-administration with one or more other drugs) and dosage schedule of L104EA29Y-Ig, and the disclosures of these patents are herein incorporated by reference.

Any suitable method or route can be used to administer the antibody polypeptide or the pharmaceutical composition. Routes of administration include, for example, oral, intravenous, intraperitoneal, subcutaneous, or intramuscular administration. A therapeutically effective dose of administered antibody polypeptide(s) depends on numerous factors, including, for example, the type and severity of the immune disease being treated, the use of combination therapy, the route of administration of the antibody polypeptide(s) or pharmaceutical composition, and the weight of the patient. A non-limiting range for a therapeutically effective amount of a domain antibody is about 0.1 to about 30 mg/kg, or about 2 to about 30 mg/kg, or about 20 to about 30 mg/kg, relative to the body weight of the patient. A therapeutically effective amount of a domain antibody can be about 20 mg/kg. A therapeutically effective amount of a BMS2h-572-633-CT-L2 (SEQ ID NO: 1) can be about 0.1 to about 30 mg/kg, or about 2 to about 30 mg/kg, or about 20 to about 30 mg/kg, or about 20 mg/kg. A therapeutically effective amount can be administered intravenously. The therapeutically effective amount can be administered on a weekly basis for the duration of the treatment regimen. The duration for the treatment regimen can vary. The duration can be about 70 days long. The domain antibody can be administered with (simultaneously, sequentially or co-formulated with) an immunosuppressive/immunomodulatory and/or anti-inflammatory agent, such as a CTLA4 mutant molecule (e.g., belatacept). The immunosuppressive/immunomodulatory and/or anti-inflammatory agent can be administered at about 20 mg/kg. Representative models are described below and in the examples.

Renal transplant patients can have one or more of several immunosuppressive agents administered during the course of their treatment. These can include glucocorticoids. Immunosuppressive agents also include small molecule drugs such as immunophilin-binding drugs (e.g., calcineurin inhibitors such as cyclophilin-binding drugs including cyclosporine and ISA(TX)247; FKBP12-binding drugs such as tacrolimus and modified-release tacrolimus; and target-of-rapamycin inhibitors such as sirolimus and everolimus), nucleotide synthesis inhibitors (such as purine synthesis inhibitors (IMPDH) such as mycophenolate mofetil, enteric-coated mycophenolic acid, and mizoribine; pyrimidine synthesis inhibitors (DHODH) such as Lefunomide and FK778), antimetabolites (such as azathioprine) and sphingosine-1-phosphate-receptor antagonists (such as FTY720). Immunosuppressive agents can also include protein drugs such as (a) depleting antibodies (e.g., against T cells, B cells, or both and can include a horse or rabbit anti-thymocyte globulin, mouse monoclonal anti-CD 3 antibodies such as muromonab-CD3, humanized monoclonal anti-CD52 antibodies (alemtuzaumab), B cell depleting monoclonal anti-CD20 antibodies (e.g., rituximab), and intravenous immune globulin. For a review of these drugs see Halloran, “Immunosuppressive Drugs for Kidney Transplantation,” New Engl. J. Med. 351: 27152729 (2004).

Contemplated is the combination of BMS2h-572-633-CT-L2 (SEQ ID NO: 1) alone in a monotherapy in a range from 2 mg/kg to 30 mg/kg. Alternatively, BMS2h-572-633-CT-L2 (SEQ ID NO: 1) can be administered in a combination therapy with a CTLA4 mutant molecule, such as L104EA29Y-Ig (Belatacept). Belatacept can be administered in combination therapy in an amount of about 10 mg/kg, 15 mg/kg, 20 mg/kg, 25 mg/kg, or 30 mg/kg (or any integer amount inbetween).

Alternatively, BMS2h-572-633-CT-L2 (SEQ ID NO: 1) can be administered in combination with an anti-CD28 Dab. A preferred anti-CD28 dAB is BMS-931699, which comprises the variable domain BMS1h-239-891 (D70C) (SEQ ID NO: 26) and is PEGylated. BMS1h-239-891 (D70C) is described in, for example, U.S. Pat. No. 8,168,759 entitled “Compositions Monovalent for CD28 Binding and Methods of Use.” The anti-CD28 dAB can be administered in an amount from 1 mg/kg to about 10 mg/kg, and for example at about 3 mg/kg.

4. Allograft Rejection In Vivo Models

The ability of antibody polypeptides of the disclosure to antagonize CD40L can be tested in one of several available in vitro or in vivo model systems. Appropriate human, animal, and cell model systems are described below. Further cell assay systems are described in the examples.

Targeting the CD40-CD40L pathway has long been of much interest for prevention of rejection of solid organ transplants (SOT), particularly in light of the promising data from numerous published transplant studies in non-human primates. It has been demonstrated that reduced CD40L expression on ex vivo activated CD4+ T lymphocytes correlates with excellent renal allograft function. Lederer et al., Int. Arch. Allergy Immunol. 133: 276-284 (2004). Furthermore, several studies have demonstrated that anti-CD40L mAbs can both prevent and reverse acute allograft rejection in primates. For example, Kirk et al., Proc. Natl. Acad. Sci. USA 94: 8789-8794 (1997) reported that, in rhesus monkeys transplanted with renal allografts, anti-CD40L mAb 5C8 alone or in combination with CTLA4-Ig significantly prolonged rejection-free survival. The CD40L-specific mAb hu5c8 alone also allowed for allogeneic islet engraftment and long-term insulin independence in rhesus monkeys that were transplanted an adequate number of viable pancreatic islets. Kenyon et al., Proc. Natl. Acad. Sci. USA 96: 8132-8137 (1999). Preston et al., Amer. J. Transplantation 5: 1032-1041 (2005) performed renal transplants in MHC (major histocompatibility complex)-mismatched rhesus monkeys and treated the recipients with combinations of CD40L-specific mAb IDEC-131, and/or sirolimus, and/or pre-transplant donor-specific transfusion. IDEC-131 was highly effective in preventing renal allograft rejection in primates. In cynomolgus monkeys that underwent renal allotransplantation, treatment with anti-CD40L mAb ABI793 effectively prevented graft rejection. Schuler et al., Transplantation 77: 717-726 (2004). In addition to preventing allograft rejection, CD40L-specific mAbs induced donor specific tolerance in primate transplant models. Preston et al., Amer. J. Transplantation 5: 1032-1041 (2005); Kenyon et al., Proc. Natl. Acad. Sci. USA 96: 8132-8137 (1999).

In pediatric human patients that were undergoing acute graft rejection after liver or small-bowel transplantation, a correlation was observed between the expression of CD40L on CD8+ T cells and the risk of transplant rejection. Ashokkumar et al., Amer. J. Transplantation 9: 179-191 (2009) and Ashokkumar et al., Surgery 146: 166-173 (2009). Similarly, in adult patients that were undergoing allograft rejection following liver or renal transplantation, histological analysis demonstrated an association between CD40L expression and acute or chronic rejection. Bartlett et al., Amer. J. Transplantation 3: 1363-1368 (2003) and Biancone et al., Nephrol. Diall. Transplant. 13: 716-722 (1998).

Several studies support targeting CD40L over CD40 to achieve better efficacy in transplantation. For example, graft survival is longer and more durable when CD40L is selectively blocked, compared to CD40. Gilson et al., J. Immunol. 183: 1625-35 (2009). Furthermore, recent data suggest that CD40L blockade may enhance induction of Tregs and/or suppressor cells to promote graft survival. Garcia et al., J. Clin. Inv. 120: 2486-96 (2010). Also, blockade of CD40L, but not CD40, has demonstrated induction of long-lived immunological tolerance resulting in indefinite graft survival, particularly when combined with blockade of the B7 pathway. Kenyon et al., Proc. Natl. Acad. Sci. USA 96: 8132-8137 (1999); Kawai et al., Amer. J. Transplantation 4: 1391-1398 (2004); Preston et al., Amer. J. Transplantation 5: 1032-1041 (2005); Adams et al., J. Immunol. 174: 542-50 (2005). The synergy of blocking CD40-40L and B7-CD28 pathways in enhancing graft survival is especially important, because it presents the presently disclosed domain antibodies as a natural choice for combination with Belatacept (L104EA29Y-Ig) for solid organ transplants (SOT).

### Exemplary Amino Acid Sequences

Representative anti-human CD40L VH domain amino acid sequences useful for the antibody polypeptides are disclosed in U.S. Provisional No. 61/955,588 in Table 1 therein. Representative nucleic acids that encode the VH domain sequences of Table 1 are set forth in Table 2 of U.S. Provisional No. 61/955,588.

As well known in the art, multiple codons can encode the same amino acid. Nucleic acids encoding a protein sequence thus include nucleic acids having codon degeneracy. The antibody polypeptides disclosed in U.S. Provisional No. 61/955,588 specifically bind CD40L. They were made using the reiterative initial/primary screening as described in detail in co-assigned U.S. Pat. No. 8,895,010, issued Nov. 15, 2014, titled “ANTIBODY POLYPEPTIDES THAT ANTAGONIZE CD40L.”

### EXAMPLES

### Example 1

### dAb Selection for Clone BMS2h-572

Three rounds of selection using decreasing concentrations of antigen (300 nM at round 1; 30 nM at round 2; 3 nM at round 3) were performed in parallel against biotinylated (1.42 moles biotin/mole trimer) human isoleucine zipper-CD40L (IZ-hCD40L) provided by Bristol-Myers Squibb. Phage from the naïve 4G and 6G Domantis dAb libraries were combined into the pools a) to h) indicated below before initiating selections:

a) 4G VH CDR3 lengths between 7-9 amino acids.

b) 4G VH CDR3 lengths between 10-12 amino acids.

c) 4G VH CDR3 lengths between 13-15 amino acids.

d) 4G VK

e) 6G VH CDR3 lengths between 7-9

f) 6G VH CDR3 lengths between 10-12

g) 6G VH CDR3 lengths between 13-15

h) 6G VK

Each round of selection involved adding the desired concentration of biotinylated CD40L to a mixture of phage (from one of the naïve library pools indicated above, or subsequent selection output phage) in 1000 μl of 2% MPBS (Phosphate Buffered Saline containing 2% (w/v) Marvel [Premier Foods, UK]) and incubating at room temperature for 1 hour by mixing end-over-end. The biotinylated antigen phage complex was then captured by adding 100 μl of resuspended Dynabeads® M-280 Streptavidin [Invitrogen, UK] (rounds 1 and 3) or 50 μl of M-280 tosyl-activated Dynabeads® (Invitrogen) that had been coupled with NeutrAvidin [Thermo Fisher Scientific, UK] (round 2) and incubated for 5 minutes with mixing end-over-end at room temperature. The Dynabeads® were then recovered using a KingFisher magnetic separator [Thermo Fisher Scientific, UK] and washed 7× with 1 mL PBST (PBS containing 0.1% (v/v) polyoxyethylenesorbitan 20 monolaurate [Sigma-Aldrich, UK]) followed by 1×1 mL PBS (phosphate buffered saline). Bound phage retained on the washed Dynabeads® were eluted by incubation with 500 μl of trypsin-PBS (50 μl of 10 mg/ml trypsin [Sigma-Aldrich, UK] dissolved in 50 mM Tris-HCl pH 7.4, 1 mM CaCl₂ added to 450 μL PBS). The phage-containing solution was recovered and 250 μL used to infect 1.75 mL of logarithmic growth phase E. coli TG1 (at an OD₆₀₀ of 0.4) for 30 minutes at 37° C. The E. coli TG1 phage infected culture was centrifuged at 11,600×g in a micro centrifuge for 1 minute and the resulting cell pellet resuspended in 1 mL 2×TY (16 g Tryptone, 10 g Yeast Extract and 5 g NaCl in 1 litre, autoclaved for 15 minutes at 121° C.) and plated onto a 9 cm Petri dish containing TYE media supplemented with 15 μg/ml tetracycline. The plates were incubated overnight at 37° C. then 2 ml of 2×TY supplemented with 15% glycerol was added to each plate and cells loosened with a glass spreader and mixed thoroughly. Fifty microliters of the scraped bacteria was used to inoculate 50 ml of 2×TY supplemented with 15 g/mL tetracycline and grown overnight at 37° C. with shaking at 250 rpm. The overnight culture was centrifuged at 3,300 g for 15 min to pellet the bacteria. To precipitate phage, 10 ml PEG/NaCl (20% Polyethylene glycol 8000, 2.5 M NaCl) was added to 40 ml supernatant. The phage/PEG solution was mixed and left on ice for 1 h, then spun at 3,300 g for 30 min at 4° C. and the supernatant discarded. The pellet was resuspended in 2 ml PBS and spun at 11,600×g for 10 min in a microcentrifuge to remove the remaining bacterial debris. The resultant supernatant containing phage was then used for the next round of selection against the appropriate concentration of biotinylated IZ-hCD40L.

### Phape ELISA

Monoclonal phage ELISAs were carried out following selection rounds 2 and 3. All washes were performed using 3 washes of 250 μl PBST followed by 3 washes of 250 μl PBS. The plates were coated overnight at 4° C. with 50 μl/well of 1 μg/ml IZ-hCD40L in PBS. The plates were washed and then blocked with 2% MPBS (modified phosphate buffered saline) for 1 hour at room temperature. The plates were washed and 25 μl/well phage supernatants added to an equal volume of 2% MPBS and incubated for 1 hour at room temperature. The plates were washed and bound phage detected with 50 μl/well anti-M13-HRP (horseradish peroxidase) conjugate [GE Healthcare, UK] diluted 1:5000 in 2% MPBS and incubated for 1 hour at room temperature. The plates were washed and the ELISA developed using 50 μl/well SureBlue 1-Component TMB MicroWell Peroxidase solution [KPL Inc, USA]. The colorimetric reaction was stopped by the addition of an equal volume of 1 M HCl and the ELISA plate read at 450 nm. Specific phage were identified by comparison to wells that were not coated with antigen but otherwise identically treated.

### Recovery of dAb Genes from pDOM4 Plasmid

The dAb V-genes from round 2 and 3 outputs were recovered by SalI and NotI restriction enzyme digestion of the phage vector pDOM4 and ligated into a SalI and NotI double digested pDOM5 expression vector.

### Soluble dAb ELISA

Binding dAbs were identified as follows. Ninety-six individual colonies containing dAb V-genes cloned into the soluble dAb expression vector pDOM5 were picked from each output into 200 μl Terrific Broth (TB) containing OnEx Autoinduction media [Novagen, UK] and incubated overnight at 37° C. with shaking at 250 rpm in Costar 96 Well Cell Culture Clusters [Corning Incorporated, USA] sealed with a gas permeable adhesive plastic strip. The cultures were centrifuged to pellet the cells and the supernatants assayed by antigen binding ELISA for dAbs that bound to IZ-hCD40L. MaxiSorp 96 well immunoplates [Nunc, USA] were coated overnight at 4° C. with 50 μl/well of 1 μg/ml IZ-hCD40L in PBS. All washes were as described for the phage ELISA. The plates were blocked for 1 hour at room temperature with 200 μl of PBS containing 1% Tween 20. The ELISA plate was washed and dAb-containing culture supernatant clarified by centrifugation at 1,800×g for 10 min at 4° C., then added to the ELISA plate (30 μL/well) to which was added an equal volume of PBST. The plates were incubated for 1 hour at room temperature and then washed. Bound dAb was detected by adding 50 μl/well 9E10 [anti-myc IgG, Sigma-Aldrich, UK] diluted 1:2000 in PBST and incubating for 1 hour at room temperature; the ELISA plate was then washed and 50 μl/well anti-mouse Fc-HRP [Sigma-Aldrich, UK] diluted 1:2000 in PBST added and incubated for 1 hour at room temperature. The plates were washed and the ELISA developed by adding 50 μl/well SureBlue 1-Component TMB MicroWell Peroxidase solution [KPL Inc, USA] and the colour allowed to develop. The colorimetric reaction was stopped by the addition of an equal volume of 1 M HCl and the ELISA plate read at 450 nm. Antigen binding dAbs were identified by comparison of the signal intensity from IZ-hCD40L wells with control wells not containing antigen.

### Example 2

### Identification of Clone BMS2h-572-6

The BMS2h-572 dAb was subjected to error-prone affinity maturation to generate the BMS2h-572 lineage. This was performed using random mutagenesis where on average 3.6 amino acid changes were introduced per dAb. Phage libraries (average size 6×10⁸) were selected using biotinylated monomeric and trimeric human CD40L with alternating streptavidin/neutravidin bead capture of the antigen (as described). Three rounds of selections using decreasing concentrations of antigen (100 nM at round 1; 10 nM at round 2; 1 nM at round 3) were performed. Sequencing was used to monitor diversity following each selection round. Selection outputs (round 2 selected on CD40L trimer for BMS2h-572) were sub-cloned into soluble expression vector pDOM13 (no C terminal tag) (as described) and screened as monoclonal bacterial micro-culture supernatants by BIAcore for improved off-rates compared to parental clones on both monomeric and trimeric CD40L. Identified improved variants were DNA sequenced and unique dAbs expressed, purified and then assayed using the BMS2h bead RBA as well as cellular CD40L driven assays (as described). Activities of these dAbs are listed in TABLE 1 below.

### Formatting BMS2h-572-6 as a Fc Fusion

BMS2h-572-6 dAb was cloned into pDOM38 vector containing Fc tail derived from human IgG1 to create DMS0502. BMS2h-572-6 dAb was also cloned into the pDOM38 vector containing Fc tail derived from human IgG4 to create DMS0505. The constructs were transiently expressed in HEK293 cells and the proteins were purified using Protein A. Purified Fc fusions were analysed by Biacore for binding to monomeric and trimeric CD40L as well as in various cell assays (as described).

### Identification of Clones BMS2h-572-608, BMS2h-572-614 and BMS2h-572-619

BMS2h-572-6 dAb was subjected to affinity maturation using a doped oligo approach. Four doped libraries were constructed for this dAb:
Library 1-5 residues in CDR1 diversified
Library 2-6 residues in CDR2 diversified
Library 3-13 residues in CDR2 diversified
Library 4-7 residues in CDR3 diversified

In each library, diversification was performed using nnS codons where n retained a large fraction of the parent base (85%) and split the rest between the equimolar amounts of the remaining three bases (5% each) and S stood for G or C. Phage libraries (average size 8×10⁸) were selected using biotinylated monomeric and trimeric human CD40L with alternating streptavidin/neutravidin bead capture of the antigen (as described). Libraries 2 and 3 were pulled together during the selection process. Three rounds of selections using decreasing concentrations of antigen (50 nM at round 1; 5 nM at round 2; 1 nM at round 3 with 200 fold excess of competitor—non biotinylated CD40L trimer) were performed. Sequencing was used to monitor diversity following each selection round. Selection outputs (rounds 2 and 3) were sub-cloned into soluble expression vector pDOM13 (no C terminal tag) (as described) and screened as monoclonal bacterial micro-culture supernatants by BIAcore for improved off-rates compared to parental clones on both monomeric and trimeric CD40L. Identified improved variants were DNA sequenced and unique dAbs expressed, purified and then assayed using the BMS2h bead RBA as well as cellular CD40L driven assays (as described). As a result, mature dAbs BMS2h-572-608, BMS2h-572-614 and BMS2h-572-619 were identified.

### Construction of Clone BMS2h-572-633

Sequence analysis revealed that all of the amino acid differences between BMS2h-572-608 and the parental dAb BMS2h-572-6 were located in CDR1 and the differences between BMS2h-572-614 and parental dAb BMS2h-572-6 were located in CDR3. Both matured dAbs shared CDR2 with the parental dAb BMS2h-572-6. This created an opportunity to construct a combination mutant which had CDR1 of BMS2h-572-608 and CDR3 of BMS2h-572-614. First, CDR1 region of BMS2h-572-608 was PCR amplified. Second the CDR2+CDR3 fragment of BMS2h-572-614 was PCR amplified. This was followed by SOE PCR (splice overlap extension polymerase chain reaction) assembly of the two fragments to create a combination mutant BMS2h-572-633. The assembled dAb PCR product was cloned into soluble expression vector pDOM13 (no C terminal tag), sequence verified, expressed, purified and then assayed using the BMS2h bead RBA as well as cellular CD401. driven assays (as described).

### Formatting BMS2h-572-633 as Fc Fusion

BMS2h-572-633 dAb was cloned into pDOM38 vector containing Fc tail derived from human IgG1 to create DMS0507. The construct was transiently expressed in HEK293 cells and the protein was purified using Protein A. Purified Fc fusion was analysed by Biacore for binding to monomeric and trimeric CD40L as well as in various cell assays (as described).

### Example 3

### CD40L Activity Cell Assays

Anti-human CD40L dAbs were assayed functionally for their ability to antagonize CD40L activities. The CD40L activities tested were B cell proliferation and cytokine production by hCD40L-driven activation of primary monocytes-derived dendritic cells (DCs). Unless otherwise noted, all assays were performed in RPMI media supplemented with 10% fetal calf serum (FCS). The results of various assays, described in detail below, are shown in TABLE 1 and TABLE 2.

### Soluble IZ-hCD40L-Driven Primary Human B Cell Proliferation:

1×10⁵ tonsillar human B cells were incubated with 0.6 μg/ml of IZ-hCD40L along with varying titration of dAb or mAb in a final volume of 200 μL/well in a 96-well round bottom plate. The plates were incubated at 37° C. for 72 hours following which thymidine (³H; 0.5 μci/well) was added for 6 hours. B cell proliferation was quantified based on thymidine incorporation. All assays, unless otherwise noted, were performed in RPMI media supplemented with 10% fetal calf serum (FCS).

### CHO-hCD40L-Driven Primary Human B Cell Proliferation:

CHO cells were transfected with human CD40L to generate a stable cell line expressing high levels of CD40L on the cell surface. CHO-CD40L cells were irradiated at 10,000 Rads before incubation with human B cells. 1×10⁵ tonsillar human B cells were incubated with 1×10³ CHO-CD40L cells (1:100 ratio of CHO-CD40L: human B cells) along with varying titrations of dAb or mAb in a final volume of 200 μl/well in a 96-well round bottom plate. The plates were incubated at 37° C. for 72 hours following which thymidine (³H; 0.5 μci/well) was added for 6 hours. B cell proliferation was quantified based on thymidine incorporation. All assays, unless otherwise noted, were performed in RPMI media supplemented with 10% fetal calf serum (FCS).

### Primary T Cell-Driven Human B Cell Proliferation:

T cells were isolated from human peripheral blood mononuclear cells (PBMCs) and enriched using via sheep red blood cell (SRBC) affinity. Enriched human T cells were cultured with PM-LCLs (EBV-transformed B cell line; irradiated at 10,000 Rads) at a 5:1 ratio (T:LCL) for 6 days at 37° C. to generate a population of allogeneic T cells. At day 6, the expanded T cells were isolated and irradiated at 3000 Rads, and then cultured (5×10⁴ T cells/well) with primary human tonsillar B cells (1×10⁵ B cells/well) at a 1:2 ratio in 96-well flat bottom plated coated with anti-CD3 mAb (OKT3). Varying titrations of dAbs/mAbs were added to each well; the final volume in each well was 200 μl. Test plates were incubated at 37° C. for 3 days. Human B cell proliferation was determined via the addition of thymidine (³H; 0.5 μci/well) to the cultures for the last 18 hours. All assays, unless otherwise noted, were performed in RPMI media supplemented with 10% fetal calf serum (FCS). In some instances, the supernatant was harvested and measured for the presence of IL-6.

### CHO-hCD40L-Driven Activation of Primary Human Monocytes-Derived Dendritic Cells (DCs):

Human PBMCs (peripheral blood mononuclear cells) were enriched for monocytes by depleting T cells via SRBC (sheep red blood cells) resetting. The monocyte-enriched PBMCs were cultured with 10 ng/ml GM-CSF (granulocyte macrophage colony-stimulating factor) and 5 ng/ml IL-4 in 6-well plates for six days at 37° C. The cultured plates were replenished with fresh media (with GM-CSF and IL-4) on days 2 and 5. The immature DCs (dendritic cells) were used in cell assays on day 6. 8×10⁴ immature DCs were cultured with 4=10³ CHO-hCD40L cells (irradiated at 10,000 Rads) along with varying titrations of dAbs/mAbs in a 96-well flat bottom plate. After 24 hours, supernatants were harvested and tested for the presence of various cytokines (IL-12, TNF, IL-23). DC activation was determined by the levels of cytokine production. All assays, unless otherwise noted, were performed in RPMI media supplemented with 10% fetal calf serum (FCS).

TABLE 1
Potency of Monomeric dAb Molecules in Various Primary Cell Assays

| | | | | CHO- |
|---|---|---|---|---|
| | hIZCD40L- | CHO-hCD40L- | | hCD40L- |
| | driven | driven Human B | T-B cell | driven |
| | Human B Cell | Cell | MLR | DC Activation |
| | Proliferation | Proliferation | EC50 | IL-12 EC50 |
| Clone | EC50 (nM) | EC50 (nM) | (nM) | (nM) |
| 2h572 | >7000.0 | | | |
| 2h572-6 | 208.0 ± 73.0 | >7000.0 | >7000.0 | >2000.0, |
| | | | | 608.0 ± 260.0 |
| 2h572-604 | 254.0, 354.0 | >700.0 | | 387.0 |
| 2h572-608 | 96.0 ± 19.0 | | >7000.0 | 152.0 ± 61.0 |
| 2h572-610 | 109.0 ± 34.0 | | >7000.0 | 207.0 ± 87.0 |
| 2h572-614 | 93.0 ± 53.0 | | >7000.0 | 135.0 ± 54.0 |
| 2h572-616 | 204.0, 340.0 | | >7000.0 | 608.0 ± 136.0 |
| 2h572-617 | 157.0, 189.0 | | >7000.0 | 338.0 ± 101.0 |
| 2h572-619 | 90.0 ± 62.0 | 421.0, 1496.0 | >7000.0 | 188.0 ± 41.0 |
| 2h572-622 | 301.0, 293.0 | | >7000.0 | 281.0 ± 127.0 |
| 2h572-623 | 181.0, 261.0 | | >7000.0 | 280.0 ± 73.0 |
| 2h572-630 | 103.0 ± 71.0 | | | 246.0 ± 240.0 |
| 2h572-631 | 108.0 ± 77.0 | | | 230.0 ± 200.0 |
| 2h572-632 | 117.0 ± 91.0 | | | 241.0 ± 190.0 |
| 2h572-633 | 20.0 ± 15.0 | | | 53.0 ± 60.0 |
| 2h572-634 | 31.0 ± 18.0 | | | 77.0 ± 67.0 |
| 2h572-635 | 29.0 ± 19.0 | | | 52.0 ± 26.0 |
| 2h572-9 | 324.0, 243.0 | | | >2000.0 |
| 2h572-11 | 140.0 ± 33.0 | >7000.0 | | 671.0 ± 165.0 |
| 2h572-12 | 79.0, 76.0 | | | 225.0, |
| | | | | >2000.0 |
| 2h572-14 | 134.0 ± 12.0 | >7000.0 | | 882.0 ± 310.0 |
| 2h572-15 | 168.0 ± 67.0 | >7000.0 | | 876.0 ± 391.0 |
| 2h572-22 | 357.0, 305.05 | | | |

TABLE 2
Potency of Fc-formatted Molecules in Various Primary Cell Assays

| | hIZCD40L- | CHO-hCD40L- | | | CHO-hCD40L- | CHO-hCD40L- | CHO-hCD40L- | CHO-hCD40L- |
|---|---|---|---|---|---|---|---|---|
| | driven Human | driven Human | | T-B cell | driven | driven | driven | driven |
| | B Cell Prolifer- | B Cell Prolifer- | T-B cell | MLR | DC Activation | DC Activation | DC Activation | DC Activation |
| | ation EC50 | ation EC50 | MLR EC50 | IL-6 EC50 | IL-12 EC50 | IL-6 EC50 | IL-23 EC50 | TNF EC50 |
| Clone | (nM) | (nM) | (nM) | (nM) | (nM) | (nM) | (nM) | (nM) |
| 2h572-6 Fc | 0.6 ± 0.4 | 3.0 ± 1.0 | 1.9 ± 0.7 | | 0.22 ± 0.18 | | | |
| 2h572-6 IgG1 | 1.0 ± 0.4 | 10.0 ± 5.0 | 3.1 ± 1.4 | 2.9 ± 1.7 | 0.58 ± 0.36 | | | |
| 2h572-6 IgG4 | 0.9 ± 0.2 | 11.0 ± 5.0 | 3.2 ± 1.5 | 1.3 ± 0.5 | 1.1 ± 0.5 | | | |
| 2h572-6-CT | 1.0 ± 0.5 | 6.0 ± 6.0 | 13.6 ± 9.2 | 8.1 ± 3.1 | 3.0 ± 1.9 | | | |
| Long Fc | | | | | | | | |
| 2h572-633 Fc | 3.5 ± 0.6 | 3.0 ± 3.0 | 0.15 ± 0.02 | 0.11 ± 0.02 | 0.34 ± 0.17 | | | |
| 2h572-634 Fc | 3.0 ± 0.0 | 3.5 ± 3.0 | 0.23 ± 0.08 | 0.19 ± 0.03 | 0.42 ± 0.05 | | | |
| 2h572-635 Fc | 2.0 ± 0.8 | 2.5 ± 1.0 | 0.16 ± 0.09 | 0.11 ± 0.02 | 0.445 ± 0.14 | | | |
| 2h572-619- | 1.5 ± 0.6 | 2.0 | 0.40 ± 0.1 | 0.3 ± 0.07 | 1.8 ± 1.3 | | | |
| Ctshort Fc | | | | | | | | |
| 2h572-619- | 1.6 ± 0.5 | 2.0 ± 1.0 | 0.72 ± 0.45 | 0.43 ± 0.12 | 1.4 ± 0.6 | 1.5 ± 0.36 | 1.5 ± 0.46 | 2.0 ± 0.7 |
| Ctlong Fc | | | | | | | | |
| 2h572-619- | 0.9 ± 0 | 1.0 ± 0.6 | 0.226, 0.216 | 0.1, 0.1 | 1.2 ± 0.6 | | | |
| N297Qshort | | | | | | | | |
| Fc | | | | | | | | |
| 2h572-619- | 0.98 ± 0.05 | 2.0 ± 0.0 | 0.480, 0.474 | 0.22, 0.11 | 1.1 ± 0.23 | | | |
| N297Qlong | | | | | | | | |
| Fc | | | | | | | | |
| 2h572-608- | 1.0 ± 0.05 | 2.0 ± 0.0 | | | 0.93 ± 0.4 | | | |
| N297Qshort | | | | | | | | |
| Fc | | | | | | | | |
| 2h572-608- | 2.0 ± 1.0 | 2.0 ± 1.0 | 0.468 ± 0.156 | 0.38 ± 0.06 | 1.6 ± 0.74 | | | |
| CT Long Fc | | | | | | | | |
| 2h572-614- | 2.0 ± 1.0 | 2.0 ± 0.5 | 0.283 ± 0.038 | 0.25 ± 0.02 | 1.4 ± 0.68 | | | |
| CT Long Fc | | | | | | | | |
| 2h572-633- | 3.0 ± 0.7 | 1.0 ± 1.0 | 0.174 ± 0.077 | 0.13 ± 0.07 | 1.9 ± 1.3 | 1.3 ± 0.3 | 1.2 ± 0.3 | 1.7 ± 0.43 |
| CT Long Fc | | | | | | | | |
| 2h572-633- | 5.0 ± 0.5 | 1.0 ± 0.5 | 0.161 ± 0.053 | 0.13 ± 0.04 | 2.3 ± 1.5 | 1.5 ± 0.7 | | 2.9 ± 1.3 |
| CT-Fc SP5 | | | | | | | | |
| 2h572-634- | 2.0 ± 1.0 | 1.0 ± 0.6 | 0.162 ± 0.029 | 0.13 ± 0.02 | 1.5 ± 0.91 | | | |
| CT Long Fc | | | | | | | | |
| 2h572-635- | 3.0 ± 1.0 | 2.0 ± 0.6 | 0.149 ± 0.014 | 0.13 ± 0.01 | 1.6 ± 0.93 | | | |
| CT Long Fc | | | | | | | | |

### Example 4

### Binding Kinetics and CD40L Affinity of Various Antibodies

BMS-986004 is a dimeric fusion protein, composed of a modified Fc fragment of IgG1 linked to the C-terminus of the dAb BMS2h-572-633. Surface plasmon resonance (SPR) was used to characterize the kinetics and affinity of BMS-986004 or the monovalent component domain antibody BMS2h-572-633 binding to CD40L. The BMS-986004 values were compared to those for the benchmark antibodies 5c8-IgG1 and 5c8-CT and the monovalent component 5c8 FAB fragment. The SPR experiments utilized an hCD40L construct containing an N-terminal isoleucine zipper motif (IZ-hCD40L) which facilitates the specific assembly of the CD40L molecule into the native trimeric form. A biotinylated version of IZ-hCD40L (biot-IZ-hCD40L) with equivalent binding activity was also utilized for some SPR experiments.

The monovalent BMS2h-572-633 domain antibody binds biot-IZ-hCD40L with a Kd of 7.8 nM, compared to an affinity of 5.4 nM for the monovalent 5c8 FAB fragment, TABLE 3. Because BMS-986004 is bivalent, and the IZ-hCD40L target is trivalent, the SPR binding data are influenced by avidity regardless of whether CD40L target is on the chip surface or in solution. To estimate the avidity-influenced binding affinity, the SPR data for BMS-986004 binding to a biot-IZ-hCD40L surface was fitted to a 1:1 Langmuir model, suggesting a dissociation constant of less than 1 nM, TABLE 3. Similar results were obtained for 5c8-IgG1 and 5c8-CT.

TABLE 3
IZ-hCD40L kinetic and affinity values
as determined using SPR (Biacore)

| Anti-CD40L | Temperature | | ka | kd | Kd |
|---|---|---|---|---|---|
| Ab | (° C.) | Model | (M−1s−1) | (s−1) | (nM) |
| BMS-986004 | 25 | 1:1 | 2.3E+06* | 2.6E−04* | 0.11* |
| | | Langmuir | | | |
| 2h572-633 | 25 | 1:1 | 1.0E+06 | 8.1E−03 | 7.8 |
| | | Langmuir | | | |
| 5c8-IgG1 | 25 | 1:1 | 5.4E+05* | 2.3E−04* | 0.42* |
| | | Langmuir | | | |
| 5c8-CT | 25 | 1:1 | 5.8E+05* | 1.3E−04* | 0.22* |
| | | Langmuir | | | |
| 5c8 FAB | 25 | 1:1 | 1.4E+05 | 7.6E−04 | 5.4 |
| fragment | | Langmuir | | | |

*Value is influenced by avidity due to analyte bivalency.

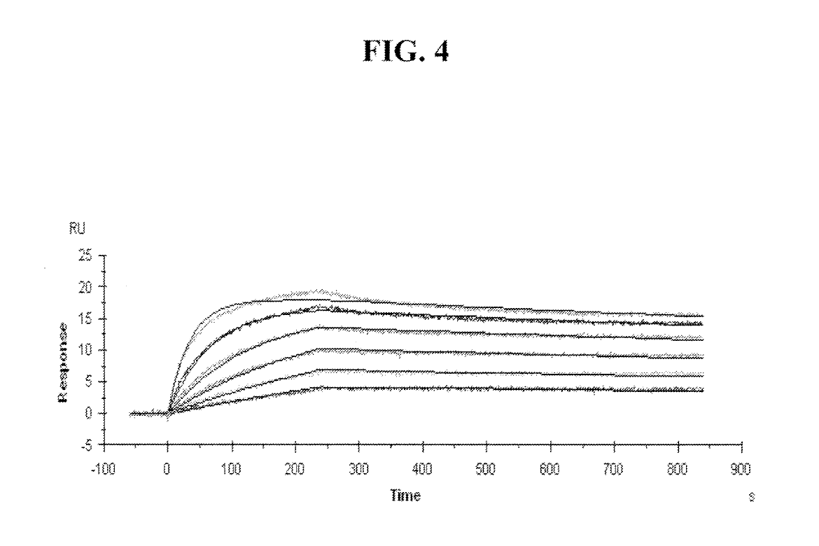

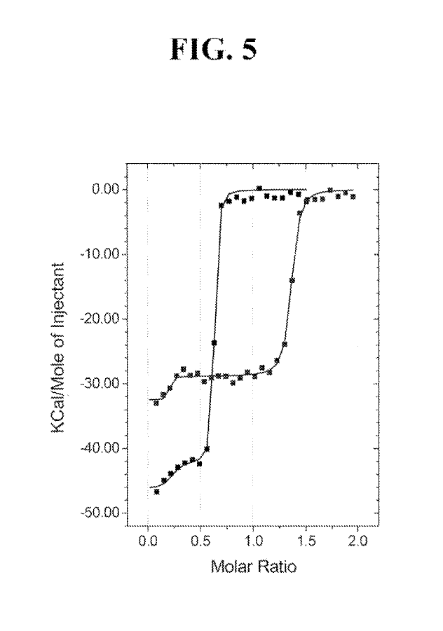

FIG. 4 shows SPR sensorgram data for the binding of 12.5-0.39 nM BMS-986004 (2:1 dilution series) to biot-IZ-hCD40L captured on a streptavidin SPR sensor chip at 25° C. Colored lines show the double-referenced sensorgram data, and black lines show the 1:1 Langmuir fit to the data, with an avidity-influenced apparent Kd value of 0.11 nM.

The affinity and thermodynamics of BMS-986004 binding to CD40L were also characterized in solution using isothermal titration calorimetry (ITC) at temperatures ranging from 15-37° C. These data suggested the presence of multiple thermodynamically distinct binding modes (FIG. 3) with Kd values for the different modes beyond the high-affinity limit of detection (Kd<2 nM) (TABLE 4), consistent with the SPR data. The affinity of the monovalent 5c8 FAB fragment for IZ-hCD40L as determined by ITC (3.5 nM) was also consistent with the value determined by SPR.

TABLE 4
IZ-hCD40L affinity as determined using ITC

| Molecule in the | Molecule in the | Kd |
|---|---|---|
| ITC syringe | ITC cell | (nM) |
| BMS-986004 | IZ-hCD40L | <2 |
| 5c8-CT | IZ-hCD40L | <2 |
| IZ-hCD40L | BMS-986004 | <2 |
| IZ-hCD40L | 5c8-CT | <2 |
| IZ-hCD40L | 5c8 FAB fragment | 3.5 |

### Example 5

### Fc Receptor Affinity of Various Antibodies

The Fc-domain of BMS-986004 (termed “CT-L2”; SEQ ID NO: 3) was engineered from a wild type IgG1 Fc domain to retain the ability to bind FcRn, but to disrupt the binding to Fcγ receptors. To confirm that the engineered molecule has the desired Fc receptor binding profile, the binding affinities of BMS-986004 for human FcRn, and the human Fcγ receptors CD64 (FcγRI), CD32a (FcγRIIa), CD32b/c (FcγRIIb/c), CD16a (FcγRIIIa), CD16b (FcγRIIIb) were measured using SPR, in comparison to 5c8-IgG1 and 5c8-CT. For these experiments, BMS-986004 was captured via the domain antibody domains on a biot-IZ-hCD40L sensor surface, and the soluble Fc receptor proteins were tested for binding to the exposed Fc domain. Likewise, 5c8-IgG1 and 5c8-CT were captured on a biot-IZ-hCD40L surface via the FAB domains, with soluble FcR binding.

BMS-986004 bound FcRn with Kd of 670 nM at pH 6.0 which is the relevant pH for binding within the endosome, TABLE 5. However, binding was significantly reduced (Kd>5000 nM) at neutral pH suggesting efficient release of from FcRn under these conditions. BMS-986004 bound CD64 with a Kd of 0.6 nM, and had a statistically weak affinity for CD32a, CD32b/c, CD16a and CD16b (Kd>3000 nM). Both 5c8-IgG1 and 5c8-CT had a similar FcRn affinity as BMS-986004. 5c8-CT, which has the identical “CT” Fc region as BMS-986004, also had a similar FcγR binding properties as BMS-986004, whereas 5c8-IgG1, which has a wild type IgG Fc domain, bound more strongly to FcγRs, TABLE 5.

TABLE 5
Fc receptor affinity as determined using SPR (Biacore).

| | | BMS-986004 | 5c8-IgG1 | 5c8-CT |
|---|---|---|---|---|
| | | Kd | Kd | Kd |
| Sample | pH | (nM) | (nM) | (nM) |
| hFcRn | 6 | 670 | 590 | 720 |
| hFcRn | 7.1 | >5000 | >5000 | >5000 |
| CD64 | 7.1 | 0.6 | <0.05 | 0.9 ± 0.4 |
| CD32a | 7.1 | >3000 | ~10⁻⁷ M* | >3000 |
| CD32b/c | 7.1 | >3000 | >3000 | >3000 |
| CD16a | 7.1 | >3000 | 240 ± 40 | >3000 |
| CD16b | 7.1 | >3000 | >3000 | >3000 |

*CD32a binding to 5c8-IgG1 was biphasic. Kd was estimated as ~10⁻⁷ M based on steady state fit to dominant binding even. This Kd is in range of literature reported KD for CD32a binding to IgG1.

### Example 6

### In-Vitro Cell-Based Assays

The potency of BMS-986004 was evaluated in various primary immune cell assays to ensure robust potency across different cell types. The primary human B cell proliferation assays were conducted two ways, as described in detail above in Example 3: (1) recombinant CD40L trimer was used to drive B cell proliferation; and (2) CHO cells expressing CD40L on the membrane (CHO-CD40L) were utilized to induce B cell proliferation. The utility of CHO-CD40L cells was particularly important to ensure that signals from membrane-bound CD40L were inhibited equally well when compared to the soluble CD40L trimer. The CHO-CD40L cells were also used to drive the activation of primary human DCs differentiated from culturing PBMC-derived monocytes in presence of GM-CSF and IL-4. Similarly, the T-B MLR (mixed leukocyte reaction) assay measured B cell activation driven by CD40L present on activated T cells. In all of the above described primary assays, BMS-986004 was equipotent to the benchmark 5c8 mAb: potencies ranged from single-digit nM to sub-nM, depending on the assay (TABLE 6).

TABLE 6
Potency of BMS-986004 in Various Primary Cell Assays

| | Trimer | CHO-CD40L | | T-B | CHO-CD40L | CHO-CD40L | CHO-CD40L |
|---|---|---|---|---|---|---|---|
| | B cell | B cell | T-B | MLR | DC Assay | DC Assay | DC Assay |
| mAb/dAb- | Assay EC50 | Assay EC50 | MLR EC50 | IL-6 EC50 | IL-12 EC50 | IL-6 EC50 | TNF-α EC50 |
| Fc | (nM) | (nM) | (nM) | (nM) | (nM) | (nM) | (nM) |
| 5c8 | 8.0 ± 3.0 | 2.0 ± 2.0 | 0.54 ± 0.37 | 0.23 ± 0.09 | 2.0 ± 1.5 | | |
| 5c8-IgG1 | 5.0 ± 1.0 | 2.0 ± 2.0 | 0.34 ± 0.13 | 0.21 ± 0.06 | 0.92 ± 0.94 | 0.73 ± 0.5 | 2.3 ± 1.3 |
| BMS- | 5.0 ± 0.5 | 1.0 ± 0.5 | 0.16 ± 0.05 | 0.13 ± 0.04 | 3.1 ± 1.6 | 1.9 ± 0.6 | 3.6 ± 1.1 |
| 986004 | | | | | | | |

### Example 7

### Assessment of Whole Blood Receptor Occupancy (RO)

A receptor occupancy method was developed to measure CD40L target engagement by BMS-986003 in cynomolgus whole blood samples and, subsequently, by BMS-986004 in human whole blood samples. BMS-986003 is a dAb which shares the same amino acid sequence as BMS-986004, except for a non-native glycine residue at its amino-terminus.

Occupancy is measured on CD4+ T cells by flow cytometry using an anti-CD40L mAb that competes for binding to CD40L with BMS-986003/BMS-986004, and is cross-reactive with human and cynomolgus CD40L. In the presence of bound dAb, the anti-CD40L detection mAb is blocked from binding to CD40L in a concentration-dependent manner, providing a measure of target occupancy. Given that basal CD40L is expressed at low levels on resting T cells in peripheral blood, RO was assessed in both unstimulated blood samples and in samples where phytohemagglutinin (PHA) was used to induce up-regulation of CD40L on the T cell surface. Binding potency curves were generated following ex vivo whole blood treatment with BMS-986003 and BMS-986004. The average EC₅₀ and EC₉₀ values obtained are shown in TABLE 7.

TABLE 7
Binding Potency of BMS-986003 and BMS-986004 on CD4+
T-cells in ex vivo Whole Blood Receptor Occupancy Assay

| | | Average | Average |
|---|---|---|---|
| | n | EC₅₀, nM | EC₉₀, nM |
| BMS-986003 | | | |
| Human (basal) | 1 | 0.9 | 3 |
| Human (PHA-induced) | 6 | 0.8 | 9 |
| Cyno (basal) | 3 | 0.6 | 3 |
| Cyno (PHA-induced) | 3 | 0.4 | 2 |
| BMS-986004 | | | |
| Human (basal) | 3 | 0.4 | 3 |
| Human (PHA-induced) | 3 | 0.7 | 5 |

The target binding potency in whole blood for BMS-986003 and BMS-986004 closely correlates between human and cynomolgus monkey. The EC₅₀ values for BMS-986003 and BMS-986004 are also similar when bound to basal and PHA-induced CD40L. Additionally, these values are comparable to those obtained in human in vitro cell based assays (see TABLE 4). Based on the measured EC₉₀ values, full target saturation in peripheral blood should be achieved at concentrations≦10 nM.

To support the preclinical PK/PD profile of BMS-986003 and BMS-986004, RO was assessed in both the cynomolgus KLH study (immunization with keyhole limpet hemocyanin) with BMS-986003 and the IV bridging study with BMS-986004. Further details of these findings can be found in Examples below.

### Example 8

### In Vivo Pharmacology

To show efficacy of a CD40L dAb in mouse disease models, a mouse CD40L dAb, 2m126-24, was formatted with mouse IgG1 Fc with D265A point mutation to further lower the Fc effector function. This mouse surrogate dAb 2m126-24-Fc shows potency comparable to BMS-986004 and MR-1, a hamster anti-mouse CD40L antibody (TABLE 8).

TABLE 8
In vitro Potency Comparison

| | | Trimer | CHO-CD40L | CHO-CD40L |
|---|---|---|---|---|
| | | B cell | B cell | DC Assay |
| | | Assay EC50 | Assay EC50 | IL-6 EC50 |
| | mAb/dAb-Fc | (nM) | (nM) | (nM) |
| Human | 5c8 | 8.0 ± 3.0 | 2.0 ± 2.0 | |
| Human | BMS-986004 | 5.0 ± 0.5 | 1.0 ± 0.5 | 1.9 ± 0.6 |
| Mouse | 2m126-24-Fc | 4.7 ± 0.9 | 0.4 ± 0.06 | 0.5 ± 0.2 |
| Mouse | MR-1 (mAb) | 1.7 ± 0.4 | 0.6 ± 0.2 | 0.6 ± 0.3 |

### Inhibition of KLH Induced Antibody Response by the Mouse CD40L dAb

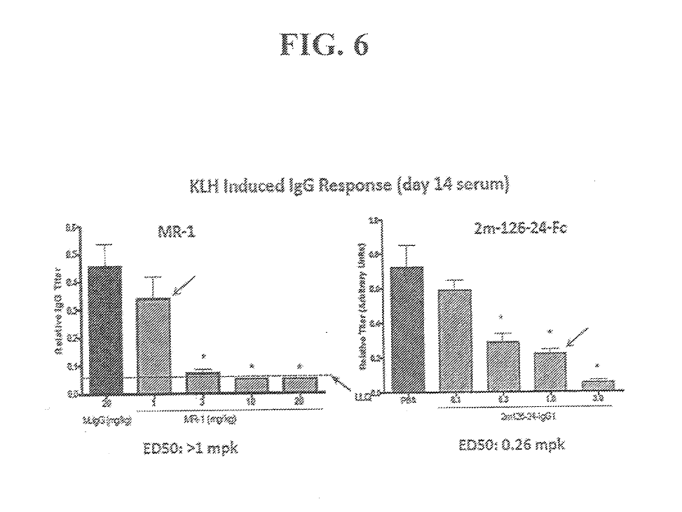

Female BALB/c mice were injected intraperitoneally (i.p.) with 250 μg KLH on day 0. Mice were dosed subcutaneously (s.c.) with MR-1 or BMS-2m-126-24-Fc at indicated doses on day −1 and day 6. Blood was collected and the serum was analyzed for anti-KLH IgM on day 7 and IgG on day 14 by ELISA. Serum from BALB/c mice collected on day 14 after immunization with KLH was pooled and used as a positive comparator, and the data is expressed as a ratio of the titer of the test serum to the titer of the pooled BALB/c serum. As shown in FIG. 6, BMS-2m-126-24-Fc demonstrated a dose dependent suppression of IgG titers with maximal effect shown at 3 mg/kg, with EC50 calculated to be 0.26 mg/kg. Both the CD40L dAb and the antibody were tested at 1 mg/kg, showing 70% versus 30% reduction in IgG response, respectively. Similar exposure of the dAb and the antibody were observed at 1 mg/kg, suggesting that the dAb is slightly more potent than the antibody at suppressing KLH-induced IgG response. In conclusion, the CD40L dAb has demonstrated at least the same level of efficacy as the anti-CD40L antibody at inhibiting a T cell dependent antibody response.

### Inhibition of TNBS-Induced Colitis by the Mouse CD40L dAb

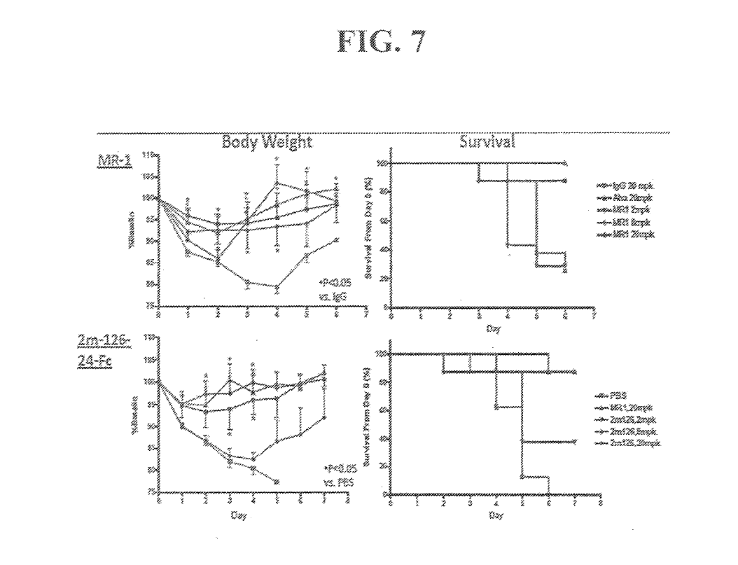

Male SJL/J mice were intrarectally administered with 2.5 mg trinitrobenzene sulfonic acid (TNBS) in 50% EtOH via a catheter inserted 4 cm distal to the anus. Mice were dosed once s.c. with MR-1 or BMS-2m-126-24-Fc at indicated doses 4 hours prior to TNBS injection. FIG. 7 presents the changes in the mean body weight and the percent survival of groups of mice treated with PBS/IgG or varying dose levels of MR-1 or the dAb. Abatacept was used as a positive control (20 mg/kg, i.p. every other day). A typical profile of TNBS-induced colitis was shown in the IgG control group: loss of body weight, peaking at day 3-4; colitis-related death occurring at day 3 and beyond; and the survived mice showing signs of recovery after day 4. Treatment with the CD40L dAb or the antibody (both tested at 2, 8 and 20 mg/kg) caused a dose-dependent inhibition of the body-weight loss and the increase in survival rate; both compounds at 8 mg/kg yielded a degree of efficacy that is comparable to that of Abatacept at 20 mg/kg. In conclusion, the mouse CD40L dAb BMS-2m-126-24-Fc has demonstrated comparable efficacy to the anti-CD40L antibody MR-1 in an acute TNBS-induced colitis model.

### Synergistic Effect Between CTLA4 Ig and the Mouse CD40L dAb in a Mouse “Heart-to-Ear” Transplant Model

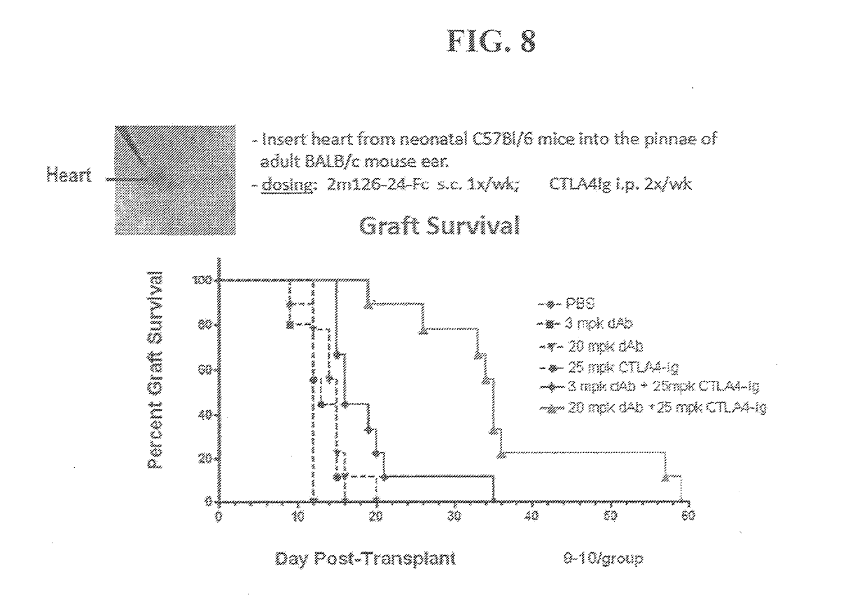

Heart grafts from neonatal (48-72 hrs) C57Bl/6 mice were implanted into a subcutaneous pocket created in the ear pinnae of BALB/c mice. Mice were treated with CTLA4-Ig (i.p. 2×/wk), BMS-2m126-24-Fc (subcutaneously, s.c. 1×/wk), or combination of both at indicated doses, with first dosing initiated the day prior to transplantation. Time to rejection was defined by the absence of cardiac contractility for three consecutive days as assessed daily by the electrocardiogram (ECG) device of allograft. As expected, without any treatment, C57BL/6 mice that received the neonatal BALB/c heart rejected the graft shortly thereafter, with median survival time (MST) of 12 days. The monotherapy with 3, 20 mg/kg of the dAb or 25 mg/kg of CTLA4-Ig had no or little impact on prolonging the survival of the allograft (MST: 12, 15 and 13 days respectively). However, in the groups treated with combination of 20 mg/kg of the dAb and 25 mg/kg of CTLA4-Ig, the graft survival was significantly prolonged showing MST of 35 days (FIG. 8). This data has provided rationale for combining CD40L dAb with belatacept in human renal transplant patients.

### Example 9

### In Vivo Non-Clinical Pharmacokinetics (PK) and Pharmacodynamics (PD)

Various in vivo studies were conducted to characterize the PK and PD of BMS-986004, BMS-986003, and a mouse CD40L dAb-Fc surrogate, BMS-2m-126-24-CT, in the nonclinical setting. The key findings are summarized below.

### ELISA to Measure BMS-986004 dAb

Enzyme-linked immunosorbency assay (ELISA)-based bioanalytical methods were developed to support the PK studies, acute and chronic efficacy studies in mice, and exploratory PK/PD studies employing cynomolgus monkeys. In all cases, whole blood was obtained and plasma prepared in the presence of EDTA, the samples were then subjected to ELISA analysis.

Plasma concentrations of BMS-986004 were measured with an ELISA assay that utilized human CD40L antigen to capture the analyte from test samples. Test samples were thawed at 4° C., mixed well and diluted 1:100 in assay diluent composed of 1×PBS, 0.05% Tween-20, and 1% BSA (PTB). Subsequent dilutions of the sample were made using 1% normal monkey plasma/PTB as diluent. This allowed the test analyte to be assayed at several dilutions (10²-10⁵) while keeping the sample matrix at 1%.

Recombinant trimeric human CD40L was obtained from Protein Structure and Science (PSS), LVL and was bound to 96 well plates at a final concentration of 2 μg/mL. Test samples, quality control (QC) samples and the standards were detected with affinity-purified rabbit anti-heavy chain (Vh) domain framework polyclonal antibody (Covance Research Products, Denver, Pa.) diluted to a concentration of 0.25 μg/ml in PTB, followed by horseradish peroxidase-labeled donkey anti-rabbit polyclonal secondary antibody (Jackson Immunoresearch, West Grove, Pa.) with substrate (TMB—tetramethylbenzidine) added, and the enzymatic reaction stopped with 1 M phosphoric acid. Absorbance was measured at a wavelength of 450 nm. The analysis of BMS-986004 in test samples was conducted using a standard curve. Standard curve calibrators prepared on the day of each run in 1% monkey plasma were used to define the dynamic range of the bioanalytical method. The range of resulting standard curve in 100% plasma was 10-1200 ng/mL. The reference standard for BMS-986004 was obtained from Biologics Process and Product Development (BPPD), HPW. The reference standard material was representative of the manufacturing batch and was used in the study protocol. Standard curves and QCs were evaluated using criteria for accuracy and precision of ≦20% which was considered to be acceptable for assay performance. Test samples were quantified using a 4-parameter logistic fit regression model weighted by reciprocal concentration (1/×) derived from the calibrators.

Performance of the QC samples, measured by the deviation of the calculated concentration from its nominal value indicated the reference material was stable in neat monkey plasma at concentrations of 30-1000 ng/ml when stored at −70° C. for over 2 months.

### Nonclinical Pharmacokinetics

TABLE 9 summarizes the PK parameters for BMS-986004, BMS-986003, and mouse BMS-2m-126-24-CT in nonclinical animal species.

TABLE 9
Single-dose PK Parameters (mean ± SD) from Two Nonclinical Animal Species

| | | | Dose | | Cmax | Tmax | AUC0-inf | T_{1/2} | CLTp | Vss | F |
|---|---|---|---|---|---|---|---|---|---|---|---|
| Species | dAb | Route | (mg/kg) | | (μM) | (h) | (μM · h) | (h) | (mL/h/kg) | (L/kg) | (%) |
| Mouse | BMS-2m- | IV | 1 | (N = 3) | — | — | 6.9 | 101 | 1.85 | 0.26 | — |
| | 126-24-CT | SC | 1 | (N = 3) | 0.063 | 24 | 10 | 100 | — | — | 100 |
| | | | 10 | (N = 3) | 0.68 | 24 | 114 | 120 | — | — | 100 |
| Monkey | BMS- | IV | 2 | (N = 2) | — | — | 40 | 106 | 0.67 | 0.067 | — |
| | 986003 | SC | 0.2 | (N = 4) | 0.019 ± 0.004 | 60 ± 72 | 4.0 ± 2.7 | 85 ± 29 | — | — | 88 |
| | | | 2 | (N = 4) | 0.22 ± 0.075 | 33 ± 43 | 29.7 ± 4.9 | 68 ± 11 | — | — | 74 |
| | | | 20 | (N = 4) | 1.48 ± 0.34 | 11 ± 9 | 175 ± 27 | 105 ± 18 | — | — | 44 |
| | BMS- | IV | 11 | (N = 4) | — | — | 241 ± 18 | 124 ± 12 | 0.59 ± 0.04 | 0.098 ± 0.01 | — |
| | 986004 | | | | | | | | | | |
| | 5c8-IgG1 | IV | 20 | (N = 4) | — | — | 1800 ± 74 | 400 | 0.074 | 0.042 | |

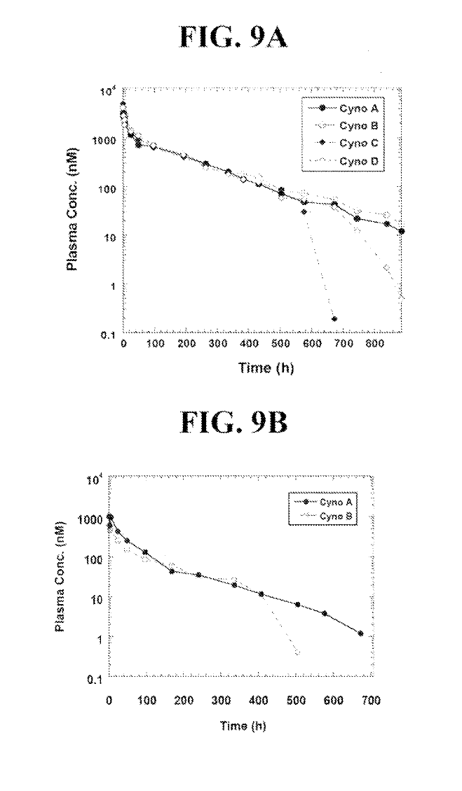

BMS-986004 and BMS-986003 exhibited comparable PK profiles in monkeys (FIG. 9A and FIG. 9B). After IV administration, the plasma concentrations of BMS-986004 and BMS-986003 exhibited a bi-exponential decline up to 504 and 408 h, respectively. Accelerated clearance was observed afterward in 50% of monkeys enrolled in both studies. Immunogenicity testing of the plasma samples collected at 38 d after BMS-986004 treatment suggested that all monkeys developed anti-drug antibody (ADA); and that the monkeys with higher ADA levels showed faster clearance. Although no immunogenicity test was conducted for the IV PK study with BMS-986003, a similar level of immunogenicity was observed in monkeys after subcutaneous dosing with BMS-986003 in the PK/PD study, suggesting both proteins were immunogenic in monkeys. The terminal half-life (T_{1/2}) of 124 and 106 h for BMS-986004 and BMS-986003 was, therefore, determined using the exposures collected up to two weeks (336 h) only. The steady-state volume of distribution (Vss) of BMS-986004 and BMS-986003 was 0.098 and 0.074 L/kg, respectively. The values are greater than the plasma volume (0.06 L/kg) but less than the volume of extracellular fluid (0.2 L/kg), suggesting that the proteins largely reside in the extracellular space. The total body plasma clearance (CLTp) of BMS-986004 and BMS-986003 was 0.59 and 0.65 mL/h/kg, respectively.

The PK parameters of BMS-986004 in monkeys were compared to those of abatacept, a similar size protein (78.5 versus 78-kDa BMS-986004, based on amino acid sequence), with the same modified human IgG1 Fc format. As expected, the parameters of BMS-986004 were nearly identical with those of abatacept (CLTp of 0.6 mL/h/kg, Vss of 0.087 L/kg, T½ of 5 d), suggesting the humans PK of BMS-986004 and abatacept is likely to be similar.

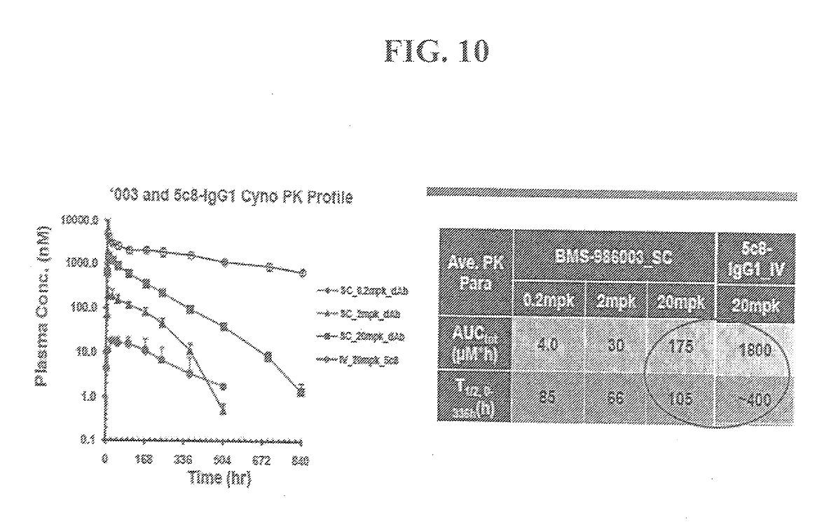

The absorption of BMS-986003 after subcutaneous (SC) administration was evaluated in the monkey PK/PD study. The monkeys were administered with BMS-986003 as single subcutaneous doses of 0 (vehicle control), 0.2, 2 and 20 mg/kg, at 24 h prior to the immunization with keyhole limpet hemocyanin (KLH), a T cell-dependent antigen. After dosing, BMS-986003 was slowly absorbed, with a Tmax ranging from 6-96 h (FIG. 10). The exposure of BMS-986003 appeared to be less than dose-proportional across all dose levels. With a dose ratio of 1:10:100, the average Cmax and AUC0-inf ratios were 1:12:80 and 1:7:44, respectively. With the exposure following the IV dose (2 mg/kg) as reference, and assuming linear PK after IV dosing, the SC bioavailability of BMS-986003 was 88%, 74%, and 44% at 0.2, 2, and 20 mg/kg, respectively. The terminal T_{1/2} was confounded by the immunogenicity observed with most of the monkeys at 2 to 5 weeks after dosing. Therefore, the T_{1/2} was estimated to be 85, 66, and 105 h at 0.2, 2 and 20 mg/kg, respectively.

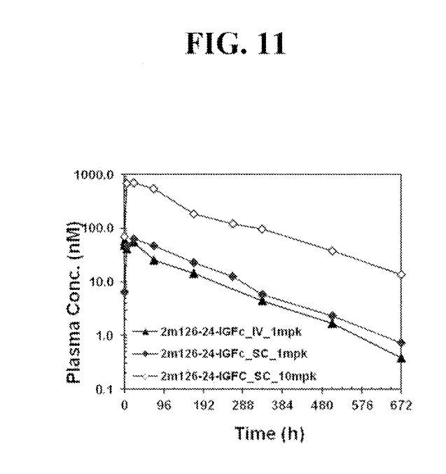

The PK of 5c8-IgG1, an anti-human CD40L monoclonal antibody used as a positive control in the PK/PD study, was evaluated after IV administration at 20 mg/kg (FIG. 11). 5c8-IgG1 exhibited 10-fold higher plasma exposures and 4-fold longer T_{1/2} when compared to BMS-986003 given SC at the same dose (TABLE 9).

The PK of the mouse surrogate dAb-Fc fusion protein, BMS-2m-126-24-CT, was evaluated in mice following single IV and SC administration (TABLE 9). After a single IV (1 mg/kg), the plasma concentrations followed a mono-exponential decline with a terminal T_{1/2} of 101 h (FIG. 11). The CLTp was 1.85 mL/h/kg; and the Vss was at 0.26 L/kg, indicating extracellular distribution. After single SC doses of 1 and 10 mg/kg, BMS-2m-126-24-CT was slowly absorbed with a Tmax of 24 h. The systemic exposures increased in a dose-proportional manner. With a dose ratio of 1:10, the Cmax and AUC0-inf increased in the proportion of 1:11. The terminal T_{1/2} was 100 and 120 h at 1 and 10 mg/kg, respectively. The ratio of the dose-adjusted exposure (AUC0-inf) after SC and IV administration was greater than 1, suggesting complete absorption after SC administration.

### Pharmacokinetic/Pharmacodynamic Modeling

The PD of BMS-986003 was measured as the suppression of anti-KLH antibody response in the PK/PD study. BMS-986003 suppressed 70% the antibody response to KLH

$$( \% response suppressed = ( 1 - \frac{{AUEC}_{0 \text{-} 1008 h IgG titers} treatedgroup}{{AUEC}_{0 \text{-} 1008 h IgG titers} vehicle group} ) * 100 )$$

at the highest dose of 20 mg/kg. Marginal (15%) and no suppression of the antibody response occurred at 2 and 0.2 mg/kg. In comparison, 5c8-IgG1 exhibited 10-fold higher plasma exposures and 4-fold longer T_{1/2} than BMS-986003 at the same dose level (20 mg/kg). As a result, 5c8-IgG1 suppressed 97% anti-KLH antibody response. To compare the in vivo potency between BMS-986003 and 5c8-IgG1, PK/PD modeling was performed using SAAM II (version 1.2.1, Seattle, Wash.). The plasma concentrations of BMS-986003 following SC administration were described using a first-order absorption kinetics coupled with a 2-compartment model, where the elimination occurred in both central and peripheral compartments. Because of complications from immunogenicity and possible nonlinear absorption, the PK data were fitted individually at each dose.

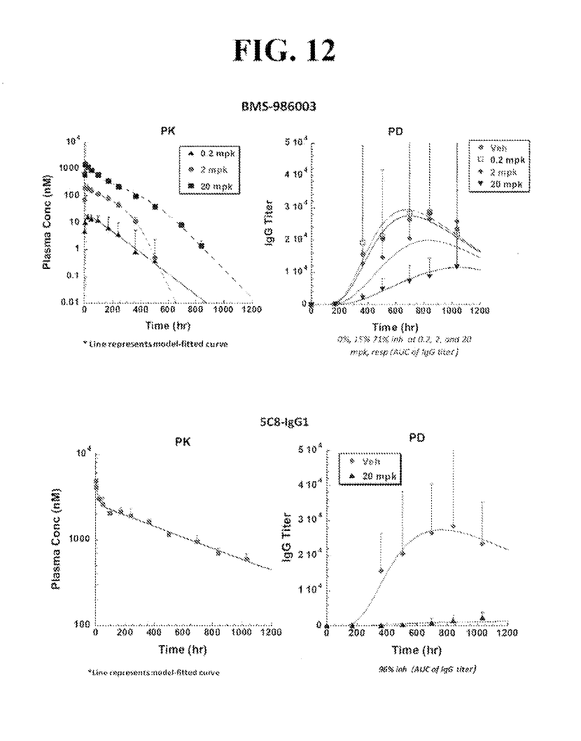

For 5c8-IgG1, a two-compartment model with central elimination was used. The anti-KLH antibody response, expressed as the average value of IgG titers, was modeled using a 6-compartment signal transduction model. The kinetics of KLH in the body was assumed to be a 1-compartment model. The inhibition of the IgG production by BMS-986003 and 5c8-IgG1 was described using an Imax model, with a maximum inhibition equal to 100%. As shown in FIG. 12, the model-fitted curves were able to describe both the PK and PD profile. The plasma IC50 of BMS-986003 and 5c8-IgG1 for the suppression of KLH-induced IgG production was estimated to be 74±14 and 60±18 nM, respectively. These results demonstrated that the potency of these two molecules was comparable in vivo.

The CD40L receptor occupancy (RO) of BMS-986004 was measured in the IV PK study. Following IV administration of 11 mg/kg, the RO of BMS-986004 on the peripheral-blood mononuclear cells (PBMC) was time- and concentration-dependent. PK/PD modeling was performed to estimate an RO EC50. The plasma concentrations were modeled using a modified two-compartment model with an additional ADA-mediated first order elimination constant introduced at 504 h after dosing; and the RO was modeled using an Emax model

$$( RO \% = \frac{E {max}^{*} {Cp}^{γ}}{EC {50}^{γ} + {Cp}^{γ}} ) .$$

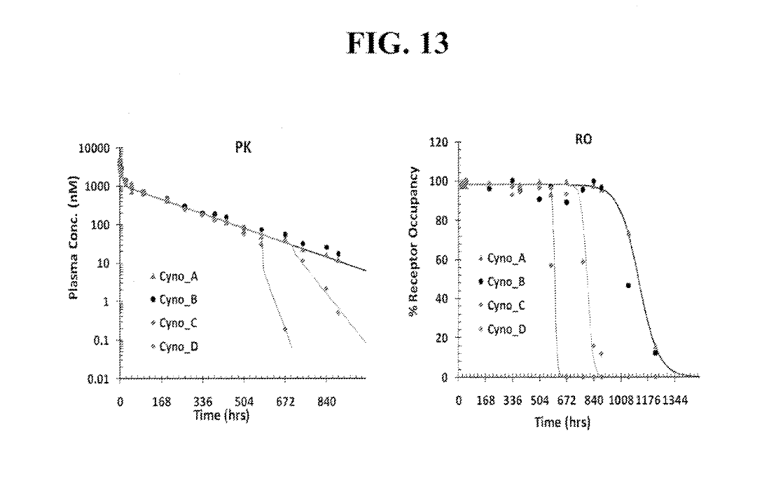

As shown in FIG. 13, the fitted curves were able to describe both exposure and RO, with an estimated RO EC50 of 3.4±0.3 nM and the γ (hill factor) of 3.1±0.1. In comparison, the RO EC50 was ˜22-fold lower than the anti-KLH antibody response IC50 of 74±14 nM, suggesting that >95% RO is required in order to achieve appreciable (>50%) anti-KLH antibody suppression.

### Example 10

### Evaluation of the Risk for TE/Thrombosis

It has been hypothesized that the TE associated with administration of the anti-CD40L monoclonal antibodies is mediated by anti-CD40L mAb-CD40L immune complex (IC)-mediated cross linking of platelets, facilitated by IC binding to FcgRIIIa, an IgG Fc receptor, causing activation and aggregation. Blocking the interaction of Fc moiety of IgG with FcgRIIIa is, therefore, expected to mitigate platelet cross linking and thrombosis. The following methods and approaches were designed to evaluate the risk of TE and/or thrombosis.

### In Vitro Platelet Activation Assays

Several in vitro assays were conducted to test the hypothesis that platelets are activated by CD40L Mab/sCD40L IC in a FcgRIIa-dependent manner. The positive control, 5c8-IgG1, was used to validate the assays prior to testing BMS-986003 and BMS-986004. Blood from human donors or mice expressing hFcgRIIa receptor on platelets were used for these studies. Platelet activation was detected by flow cytometry using antibodies against the well-validated platelet activation markers P-selectin (CD62P) and PAC-1 (activated GPIIb/IIIa). Briefly, blood was diluted 1:25 in modified Tyrodes-HEPES containing 1 mM CaCl2 to which detection antibodies and test reagents was added, incubated, and analyzed for platelet activation. Initial experiments determined that sCD40L or 5c8IgG1 alone did not activate platelets, but different immune complex ratios of 1:1 to 1:8 of 5c8:sCD40L significantly activated platelets. Subsequent experiments used 5c8-IgG1 or 5c8-mIgG2a IC, mostly at a 1:3 molar ratio of 5c8:sCD40L.

Platelet Activation by 5c8/sCD40L IC can be Blocked by Anti-FcgRIIa Antibody

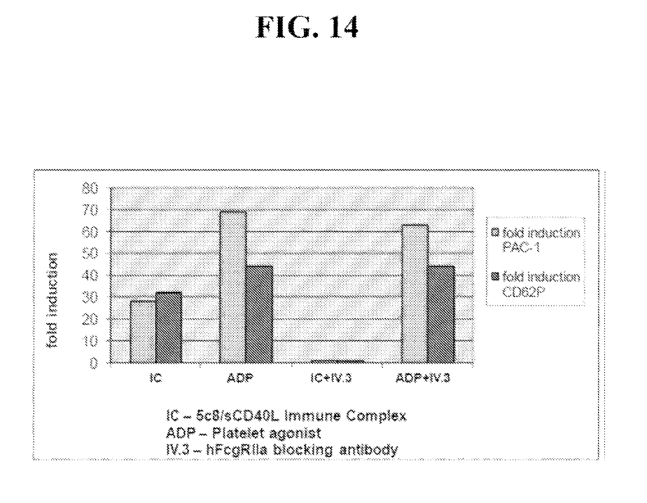

Studies were conducted with the FcgRIIa blocking antibody IV.3 to test whether activation of platelets by 5c8/sCD40L IC was indeed FcgRIIA-mediated. Blood from human donors was pre-incubated with 0.5 μg/μl of the FcgRIIa blocking antibody IV.3 prior to dilution and incubation with detection antibodies as described above. Adenosine diphosphate (ADP), a platelet activator via a different mechanism, was used as a positive control. As illustrated in FIG. 14, platelet activation by 5c8/sCD40 IC was completely blocked by IV.3, while activation by ADP was not inhibited by the blocking antibody, indicating that activation by the IC is FcgRIIa-mediated.

### Selection of Inert Fc Tails

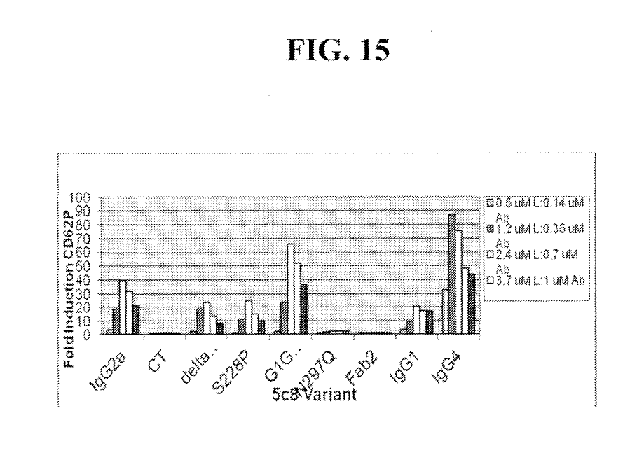

A requirement for potential candidate molecules was absence of binding to FcgRIIa to prevent potential platelet activation. Several 5c8 constructs containing different mutations derived from IgG1 (e.g., 5c8-CT and N297Q) or IgG4 (e.g., 5c8-S228P) were expressed and screened for Fc tails that did not activate platelets using different molar ratios of sCD40L to mAbs. Wild-type and most mutated constructs activated platelets except for 5c8-CT and 5c8-N297Q (FIG. 15). Absence of Fc (5c8-Fab2) also did not activate platelets further confirming that IC-platelet activation is Fc-mediated. The CT tail was chosen to format the dAb candidates BMS-986003 and BMS-986004.

### Effect of FcgRIIa Polymorphism on Platelet Activation

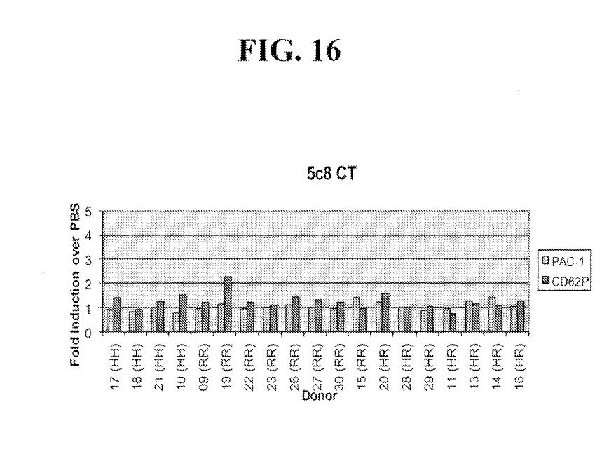

The gene for FcgRIIa is variable at codon 131, resulting in His-Arg (CAT/CGT) polymorphism. The genotype distribution in approximately 100 individuals with about equal distribution of Caucasians and African Americans was A/A (His homozygous; 14%), A/G (His/Arg heterozygous; 60%), and G/G (Arginine homozygous; 26%) for Caucasian Americans and A/A (30%), A/G (51%), and G/G (19%) for African-Americans. Reilly et al., Clin. Diagn. Lab. Immunol. 1: 640-644 (1994). Fc-dependent platelet aggregation was noted in samples from R131 individuals when treated with anti-CD9 in mIgG2 or mIgG1 Fc format, while platelets from H131 individuals aggregated only with anti-CD9 as mIgG2 format; this suggests that Fc-dependent aggregation with an IgG1 mAb could potentially segregate a patient population into low and high responders, which has previously been reported with this polymorphism. Tomiyama et al., Blood 80: 2261-2268 (1992). To address any potential differences in platelet activation with the IgG1 and CT Fc tail, 19 donors were genotyped for hFcgRIIa polymorphism and samples tested for platelet activation. The donor pool polymorphism (RR; 42%, HH; 21%, HR; 37%) was sufficient to evaluate any potential differences in platelet activation to the IgG1 format. Representative of literature reports, platelet activation with 5c8-IgG1/sCD40L IC was similar across all genotyped individuals. No activation was found with 5c8-CT/sCD40L IC (FIG. 16), suggesting no or minimal risk of increased TE in a patient population with an antibody formatted with the CT tail.

### BMS-986004: Platelet Activation in Human Blood Donors

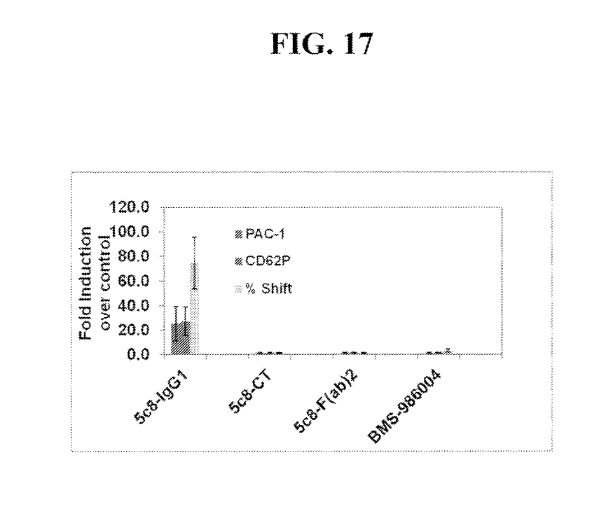

The experiments described above using 5c8, supported selection of the CT-tail as the best format for BMS-986004 (also called BMS2h-572-633-CT-L2). Blood obtained from 6 donors was treated with 5c8-IgG1, 5c8-CT, F(ab)₂, and BMS-986004. Platelets were activated by 5c8-IgG1 but not by any of the other constructs, including BMS-986004 (FIG. 17), suggesting that this dAb has no or low risk for causing platelet activation and TE in clinical studies.

BMS-986003: Platelet Activation in Blood from Mice Expressing hFcgRIIa

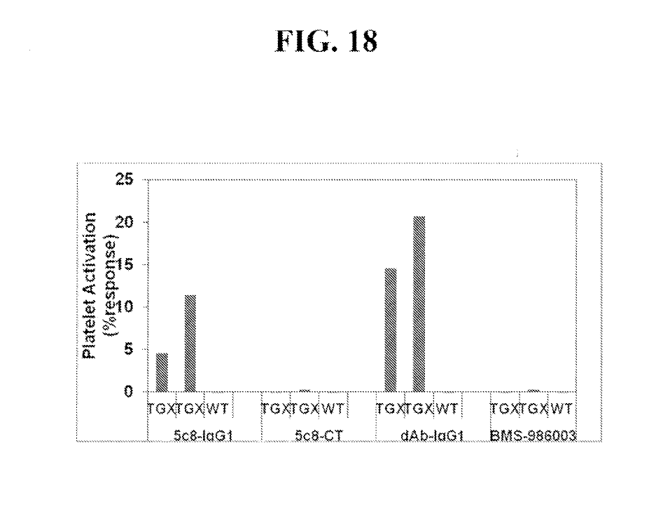

To further confirm that activation of platelets by anti-CD40L antibodies was mediated by FcgRIIa receptor, blood from transgenic mice expressing the human receptor (R131 genotype) was treated with 5c8-IgG1, 5c8-IgG2a, dAb-IgG1, 5c8-CT, and BMS-986003 (also called BMS-2h572-633-CT). Platelets were specifically activated by 5c8-IgG1, 5c8-IgG2a, and dAb-IgG1/sCD40L IC in blood from mice expressing hFcgRIIa, but not wild-type littermates. 5c8-CT and BMS-986003 did not activate platelets, further confirming a low risk for TE with the presently disclosed antibodies (FIG. 18).

### Example 11

### Immunosuppression Regimens

Transplant studies were conducted in Rhesus macaques to evaluate the efficacy of BMS-986004 alone, and in combination with belatacept, in a non-human primate renal transplant model. For these studies, the monkeys were divided into the following dose response groups and treatment regimens:

Phase 1—Part 1: Dose response for BMS-986004 only (n of monkeys=9)

Group A (High dose): BMS-986004 20 mg/kg intravenous (n=6)
Administration on post-operative day (POD) 0, 7, 14, 21, 28, 35, 42, 49, 56, 63, and 70 (weekly)
Sacrifice on POD 77

Group B (Intermediate dose): BMS-986004 10 mg/kg intravenous (n=1)
Administration on POD 0, 7, 14, 21, 28, 35, 42, 49, 56, 63, and 70 (weekly)

Group C (Low dose): BMS-986004 dAb 2 mg/kg intravenous (n=2)
Weekly administration through POD 70
   Phase 1—Part 2: Combination treatment with Belatacept (n=6)

BMS-986004 alone 20 mg/kg intravenous
Administered on POD 0, 7, 14, 21, 28, 35, 42, 49, 56, 63, and 70 (weekly)

BMS-986004 20 mg/kg intravenous+Belatacept 20 mg/kg intravenous
Administered on POD 0, 7, 14, 28, 42, 56, and 70
   Phase 2—Part 1: Dose response for BMS-986004 only (n=5)

Group A (High dose): BMS-986004 20 mg/kg intravenous (n=3)
Administered on POD 0, 7, 14, 21, 28, 35, 42, 49, 56, 63, and 70 (weekly)

Group B (Higher dose): BMS-986004 30 mg/kg intravenous (n=2)
Administered on POD 0, 7, 14, 21, 28, 35, 42, 49, 56, 63, and 70 (weekly)

Animals were followed for survival until endpoints. As demonstrated in the following Examples, some of the monkeys did not survive until the desired endpoint. These animals could not complete the planned dosing regimens. The outcomes are discussed in the following Examples.

### Example 12

### Graft Function

Transplant studies in rhesus monkeys were conducted to assess appropriate dosing for CD40L dAb BMS-986004. An assessment of BMS-986004's impact on survival and characterization of any rejection response was completed in order to understand the underlying mechanisms.

### Laboratory Assessment

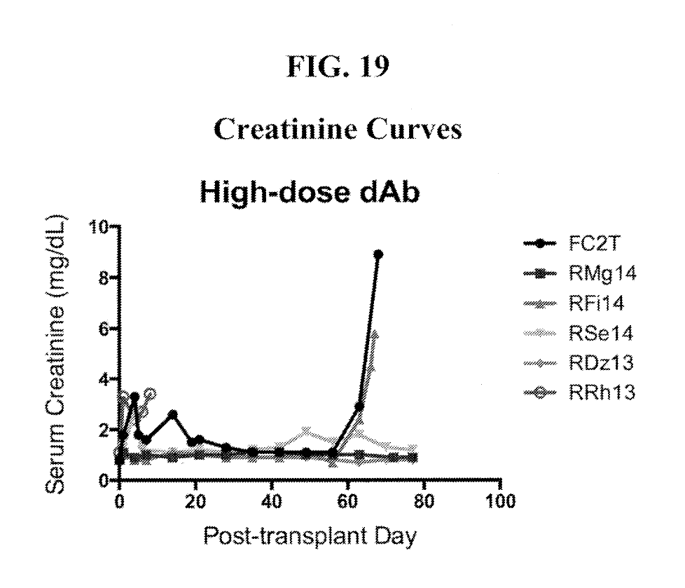

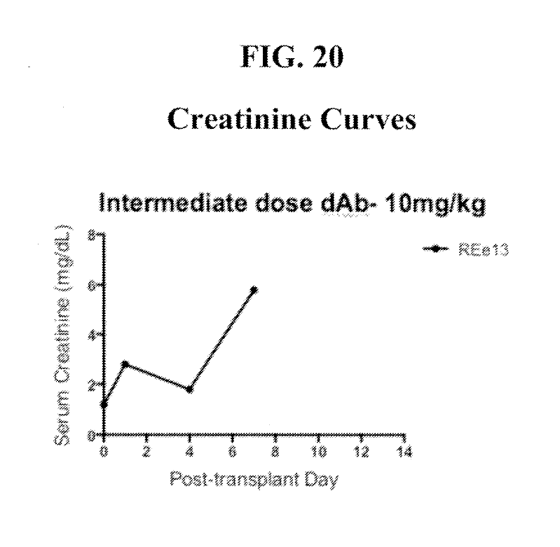

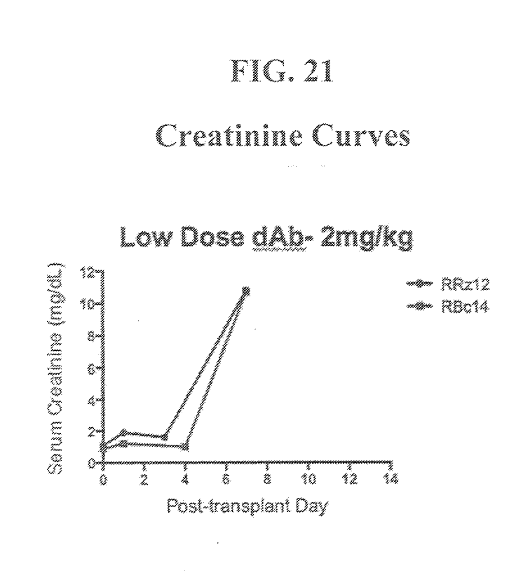

Serum creatinine studies were conducted to test allograft function in Rhesus monkeys treated with BMS-986004 over time. Allograft failure was defined as the development of renal failure sufficient to require dialysis in a clinical setting (i.e., BUN>100 mg/dL, or hyperkalemia>7.0 associated with a rising creatinine) with BUN and serum creatinine levels being used as the biomarkers for renal failure. FIGS. 19-21 show serum creatinine levels for Phase I—Part 1 renally transplanted rhesus monkeys treated with a high dose (20 mg/kg), medium dose (10 mg/kg), and low dose (2 mg/kg) of BMS-986004. The low dose and medium dose groups demonstrated a hyperkalemia>6.0 associated with a rising creatinine levels after approximately 6 days after transplant. None of the monkeys in the high dose group demonstrated a hyperkalemia>7.0 before 60 days after transplant. Recipient survival time was recorded, and the rhesus monkeys were euthanized at the time of allograft failure.

TABLE 10 below provides Phase I—Part 1 recipient survival data and clinical assessments:

TABLE 10
Recipient Survival - Low, Intermediate, and High Doses

| | Recipient | Graft Survival | |
|---|---|---|---|
| Therapy | ID | (Days) | Comments |
| Anti-CD154 | FC2T | 68 | Rejection |
| dAb alone | RMg14 | 77 | Planned SAC |
| (20 mg/kg) | RFi14 | 67 | Rejection |
| BMS-986004 | RSe14 | 77 | Planned SAC |
| | RDz13 | 77 | Planned SAC |
| | RRh13 | 8 | Rejection |
| Anti-CD154 | REe13 | 7 | Rejection |
| dAb alone | | | |
| (10 mg/kg) | | | |
| BMS-986004 | | | |
| Anti-CD154 | RRz12 | 7 | Rejection |
| dAb alone | RBc14 | 7 | Rejection |
| (2 mg/kg) | | | |
| BMS-986004 | | | |

* Planned Sac = planned sacrifice

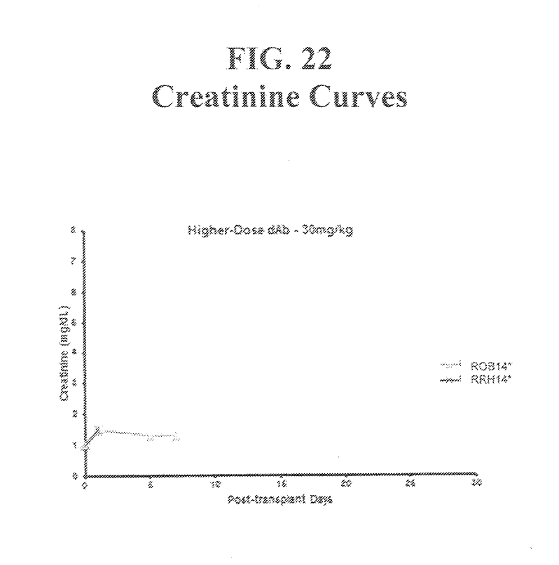

Similar experiments were conducted for Phase I—Part 2 renally transplanted rhesus monkeys treated with an even higher dose (30 mg/kg). FIG. 22 provides Creatinine Curves for the treated monkeys. TABLE 11 below provides Phase I—Part 2 recipient survival data and clinical assessments:

TABLE 11
Recipient Survival - Dose Escalation

| | Recipient | Graft Survival | |
|---|---|---|---|
| Therapy | ID | (days) | Comments |
| Anti-CD154 | ROb14* | >8 | Ongoing |
| dAb alone | RRh14* | >3 | Ongoing |
| (30 mg/kg) | | | |
| BMS-986004 | | | |

In additional studies, the following results
were observed across five recipients.

| | | | |
|---|---|---|---|
| Anti-CD154 | RRh14 | 121 | Rejection |
| dAb alone | RG14 | 103 | Rejection |
| (30 mg/kg) | RYw13 | 103 | Rejection |
| | RNu13 | 38 | Rejection |
| | Rob14 | 36 | Rejection |

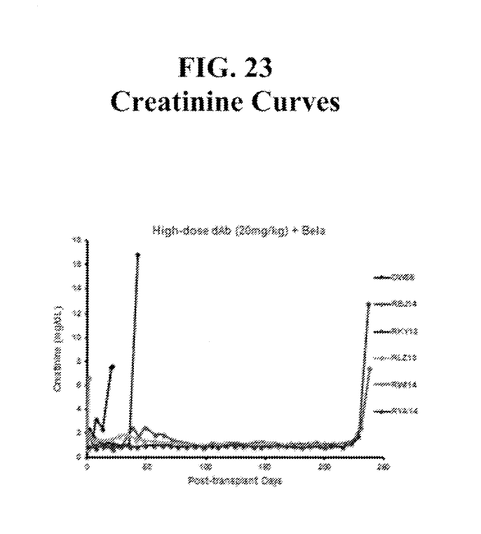

Similar studies were conducted for Phase 2 renally transplanted rhesus monkeys treated with 20 mg/kg BMS-986004 group or 20 mg/kg BMS-986004+20 mg/kg Belatacept. FIG. 23 provides Creatinine Curves for the treated monkeys. TABLE 12 below provides Phase II—Part 1 recipient survival data and clinical assessments. Last dose of anti-CD154 dAb was at Day 70 and the last dose of belatacept was at Day 168 post-graft.

TABLE 12
Recipient Survival - Combination Therapy

| | Recipient | Graft Survival | |
|---|---|---|---|
| Therapy | ID | (days) | Comments |
| Combo | RLz13 | 309 | Endpt Sac |
| Anti-CD154 | RBj14 | 238 | Rejection |
| dAb (20 mg/kg) + | RYa14 | 237 | Rejection |
| Belatacept | RKy13 | 42 | Rejection |
| | DW86 | 21 | Rejection |
| | RWi14 | 1 | Technical |

### Renal Allograft Biopsies

To further assess allograft function, the rhesus monkeys underwent a percutaneous kidney biopsy on post-transplant days 35 and 70. Preliminary histologic analysis was performed by a veterinary pathologist with expertise in renal transplant. The biopsies were characterized by standardized Banff criteria used to diagnose renal allograft rejection. The Banff criteria are defined in TABLES 13-16 below:

TABLE 13
Criteria for Acute T-cell Mediated Rejection

| Type | |
|---|---|
| (Grade) | Histopathological Findings |
| IA | Cases with significant interstitial infiltration (>25% |
| | of parenchyma affected, i2 or i3) and foci of moderate |
| | tubulitis (t2) |
| IB | Cases with significant interstitial infiltration (>25% |
| | of parenchyma affected, i2 or i3) and foci of severe |
| | tubulitis (t3) |
| IIA | Cases with mild to moderate intimal arteritis (v1) |
| IIB | Cases with severe intimal arteritis comprising >25% of |
| | the luminal area (v2) |
| III | Cases with “transmural” |

TABLE 14
Quantitative Criteria for Mononuclear Cell Interstitial
Inflammation (“i scores”)

| | |
|---|---|
| i0 | No or trivial interstitial inflammation |
| | (<10% of unscarred parenchyma) |
| i1 | 10 to 25% of parenchyma inflamed |
| i2 | 26 to 50% of parenchyma inflamed |
| i3 | More than 50% of parenchyma inflamed |

TABLE 15
Quantitative Criteria for Tubulitis (“t scores”)

| | |
|---|---|
| i0 | No mononuclear cells in tubules |
| i1 | Foci with 1 to 4 cells/tubular cross section (or 10 tubular cells) |
| i2 | Foci with 5 to 10 cells/tubular cross section (or 10 tubular cells) |
| i3 | Foci with >10 cells/tubular cross section, or the presence of at |
| | least two areas of tubular basement membrane destruction |
| | accompanied by i2/i3 inflammation and t2 tubulitis elsewhere |
| | in the biopsy |

TABLE 16
Quantitative Criteria for Intimal Arteritis (“v scores”)

| | |
|---|---|
| v0 | No arteritis |
| v1 | Mild-to-moderate intimal arteritis in at least one arterial |
| | cross section |
| v2 | Severe intimal arteritis with at least 25% luminal area lost |
| | in at least one arterial cross section |
| v3 | Transmural arteritis and/or arterial fibrinoid change and |
| | medial smooth muscle necrosis with lymphocytic infiltrate |
| | in vessel |

Results of the percutaneous kidney biopsy are included in the table below.

TABLE 17
Percutaneous Kidney Biopsy Histologic Analysis

| | ID Post- | Creatinine | | | | Overall |
|---|---|---|---|---|---|---|
| Rhesus | treatment | levels | i | t | v | Banff |
| monkey | (day) | (mg/dL) | score | score | score | grade |
| RFi14 | 36 (Bx*) | 0.9 | 2 | 2 | 0 | ACR IA |
| | 67 (Sac*) | 5.8 | 3 | 3 | 0 | ACR IB |
| RMg14 | 36 (Bx) | 1.1 | 1 | 0 | 0 | Borderline |
| | 72 (Bx) | 0.9 | 1 | 1 | 0 | Borderline |
| | 77 (planned Sac) | 0.9 | 0 | 1 | 0 | Borderline |
| FC2T | 35 (Bx) | 1.1 | 1 | 1 | 0 | Borderline |
| | 68 (Sac) | 8.9 | 2 | 2 | 0 | ACR IA |
| RSe14 | 34 (Bx) | 1.2 | 2 | 1 | 0 | Borderline |
| | 69 (Bx) | 1.3 | 1 | 2 | 0 | Borderline |
| | 77 (planned Sac) | 1.2 | 2 | 2 | 1 | ACR IIA |
| RDz14 | 36 (Bx) | 0.9 | 0 | 1 | 0 | Borderline |
| | 71 (Bx) | 0.8 | 2 | 2 | 0 | ACR IA |
| | 77 (planned Sac) | 0.8 | 1 | 2 | 0 | Borderline |
| RRh13 | 8 (sac) | 3.4 | 2 | 2 | 1 | ACR IA |

*Bx = biopsy;
*Sac = sacrifice

### Example 13

### Cellular Phenotyping by Flow Cytometry

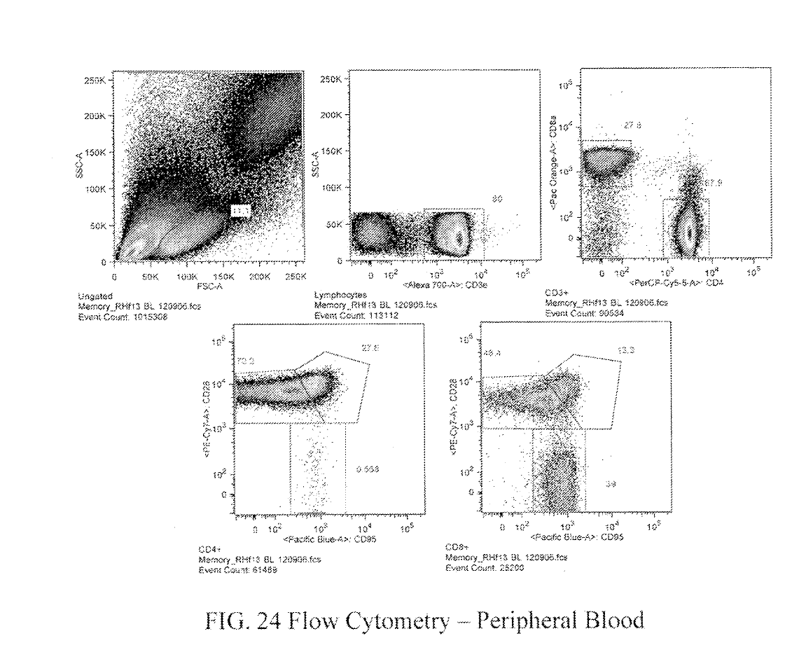

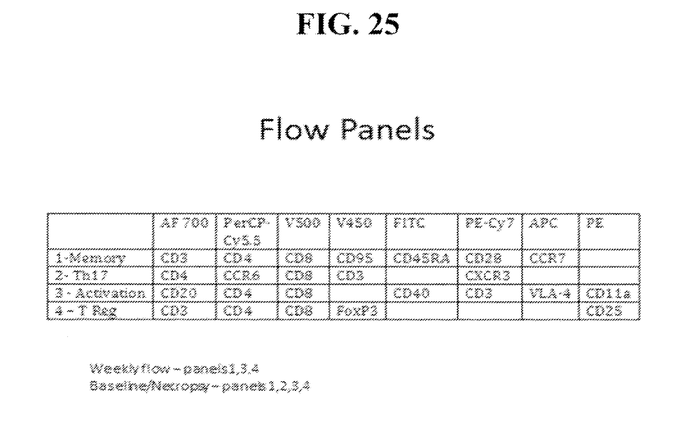

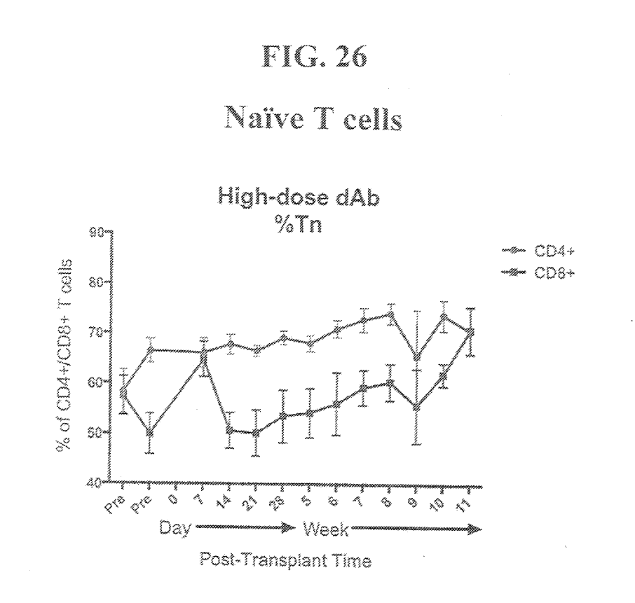

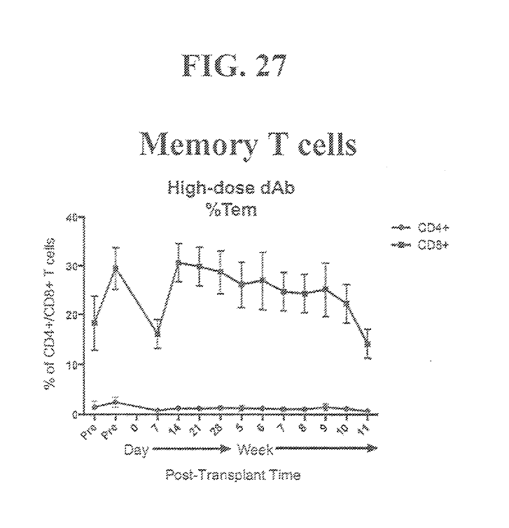

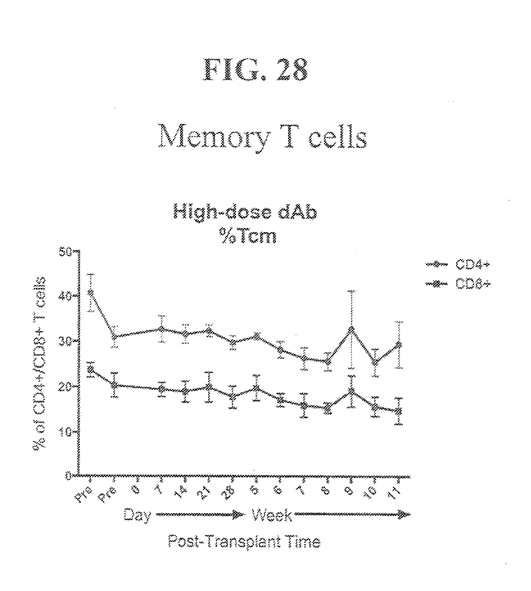

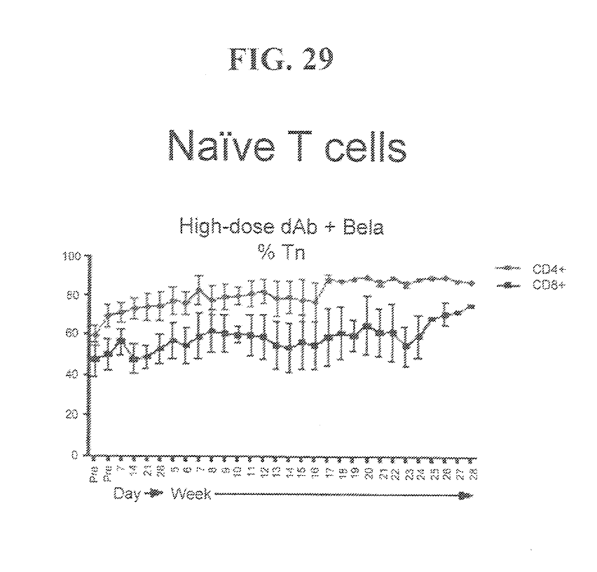

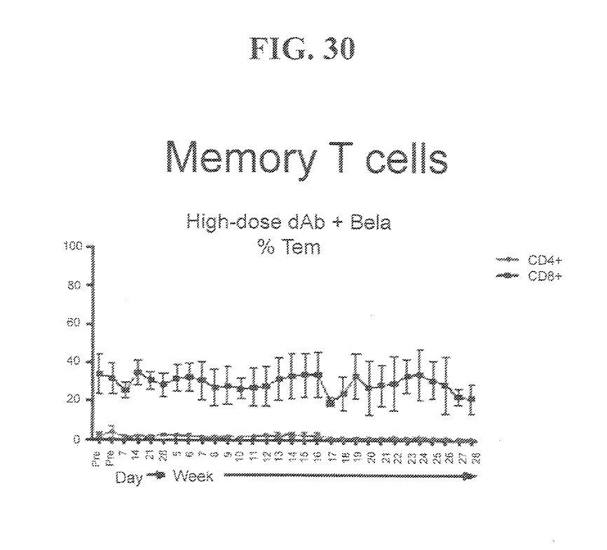

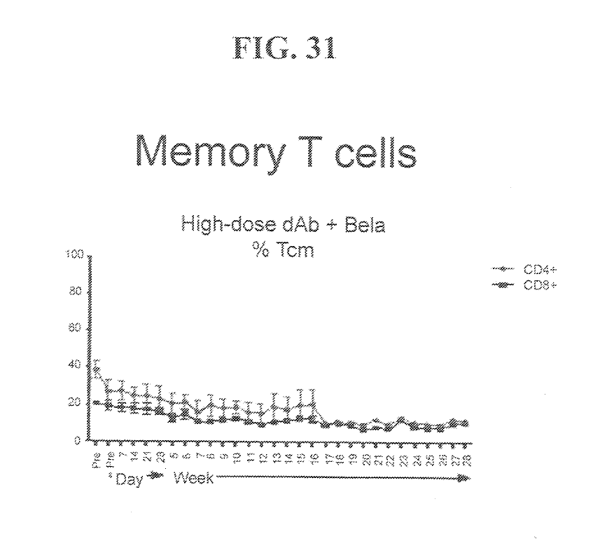

Peripheral blood samples were collected from Phase II rhesus monkeys and a cellular phenotypic analysis was performed to assess leukocyte composition (immunophenotype) and other cellular markers consistent with immune activation. Preliminary mean group T cell subset flow cytometry data for the Phase II—20 mg/kg BMS-986004 group and 20 mg/kg BMS-986004+20 mg/kg belatacept group is shown in FIGS. 24-31.

Renal allograft, spleen, lymph node, and bone marrow samples were also collected at the time of being euthanized. Allograft parenchyma is processed for extraction of tissue infiltrating cells, and is analyzed by flow cytometry and gene array expression profiling.

### Example 14

### Levels of BMS-986004 and Belatacept

Studies are conducted to determine anti-BMS-986004 and anti-belatacept levels in plasma samples that are collected from Phase I—Part 1 rhesus monkeys treated with 2 mg/kg (n=2), 10 mg/kg (n=1), or 20 mg/kg (n=6) of BMS-986004 and belatacept. Samples are obtained immediately before each dose is given on day 0 (pre-transplant); at the conclusion of the transplant (2 hrs after the pre-transplant infusion); post-transplant days 4, 7, 14, 28, and bi-weekly thereafter.

### Example 15

### Viral Load Assays

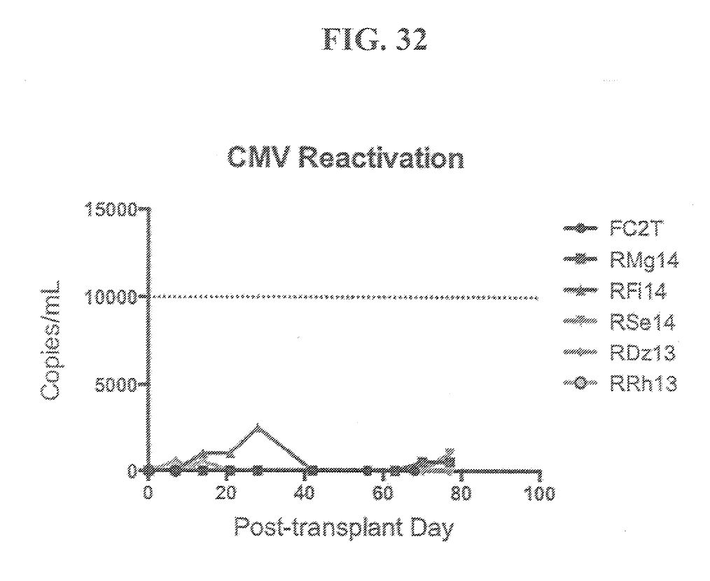

Viral reactivation has been shown to occur post-transplant when the patient is in an immunosuppressed state. Rhesus monkeys treated with BMS-986004 at 20 mg/kg were monitored for the presence of cytomegalovirus (CMV) viral reactivation using real-time PCR techniques which have been previously described. There was no evidence of cytomegalovirus (CMV) viral reactivation in any of the treated monkeys. (See FIG. 32) This data provides further support that the immune system is being adequately suppressed by BMS-986004 and that there is no reactivation of CMV.

Rhesus monkey's treated with BMS-986004 at 20 mg/kg are monitored for the presence Rhesus cytomegalovirus (RhCMV), simian virus 40 (SV40), and lymphocryptovirus (LCV) by analyzing Rhesus monkey whole blood.

### Example 16

### Assessment of Thromboembolic Potential

Rhesus monkey plasma samples from various time points are analyzed for D-Dimer, fibrinogen, and anti-thrombin levels, as well as PT/aPTT (prothrombin time/activated partial thromboplastin time). Weekly platelet counts are recorded from complete blood counts (CBC). Platelet distribution width is also recorded. Platelet distribution width is an index used as a marker for the diagnosis of thromboembolism. Platelet distribution width increases due to platelet activation associated with thromboembolism.

Collection time points are initially concentrated around operative procedures and then spaced out at regular time points throughout the remaining life of the monkey. Plasma samples are collected at two pre-nephrectomy times; post nephrectomy day 1 and 7; post-transplant day 0, 1, 4, 7, 14, 21, 28, and then every 2 weeks until and including the time of euthanasia.

### Example 17

### Necropsy Evaluation

A necropsy is performed on the rhesus monkeys to ascertain if they display any thromboembolic complication-like symptoms. A standard gross examination is performed. Tissues are collected from all the monkeys, including renal allograft, adrenal gland, brain, colon, duodenum, heart, ileum, inguinal lymph node, mediastinal lymph node, jejunum, liver, lung, mesenteric lymph node, pancreas, parathyroid, skin, spleen, stomach, thymus, and thyroid tissues. These samples are collected in 10% neutral buffered formalin. Additional samples of renal allograft, heart, skin, lung, spleen, thymus, mediastinal lymph nodes and inguinal lymph nodes are collected and stored. Any grossly abnormal tissue area is also collected, along with corresponding areas of tissue from control monkeys, where possible.

Upon sacrifice, there was no gross or histologic evidence of thromboembolism (TE). Various T-cell analyses determined evidenced that protective immunity was maintained for the animals. The treatment was found to be safe and efficacious and having similar potency to previous anti-CD154 therapies.

### Example 18

### Monotherapy, Monotherapy+Conventional Therapy, and Combination Therapy

Following the same guidelines and using the same animals as described above 20 mg/kg anti-CD28 dAb alone or in some combined therapy was administered. Belatacept was administered prior to renal transplant at a dose of 10 mg/kg, then again at Day 4 post-transplant at 15 mg/kg and at days 7, 14, 18, 42, 56, 84, 112, 140 and 168 at 20 mg/kg for either the monotherapy with belatacept alone or in combination with the conventional therapy or in combination with the anti-CD28 Dab. Conventional therapy consisted of:

A) an anti-IL-2R being administered at DAY 0 and shortly thereafter;

B) 20 mg/day of Solumedrol starting from Day 0 to Day 28, being decreased to 2 mg/day Solumedrol Day 29 to Day 84, at which point Solumedrol was decreased to 1 mg/day; and

C) 15 mg mycophenolate mofetil (MMF) being administered b.i.d. (bis in die or twice a day) from Day 0 to Day 28 and then 15 mg. q.d. (quaque die or once a day) thereafter.

The results appear in Tables 18-20 for the animals administered the various therapies.

TABLE 18

| | Recipient | Graft Survival | |
|---|---|---|---|
| Therapy | ID | (days) | Comments |
| Anti-CD28 | H326 | >224 | Ongoing |
| dAb alone | H324 | >216 | Ongoing |
| (20 mg/kg) | RTg15 | >175 | Ongoing |
| | RAm14 | 53 | Rejection |
| | GB29 | 35 | Rejection |
| | RHn14 | 10 | Rejection |
| Belatacept | RHH14 | 53 | Rejection |
| alone | RRI13 | 47 | Rejection |
| | RBB144 | 29 | Rejection |
| | RDI13 | 12 | Rejection |
| | DW13 | 8 | Rejection |

TABLE 19

| | Recipient | Graft Survival | |
|---|---|---|---|
| Therapy | ID | (days) | Comments |
| Anti-CD28 | RKm14 | >196 | Ongoing |
| (20 mg/kg) + | RSk14 | >195 | Ongoing |
| Conventional | GB44 | >159 | Ongoing |
| Therapy (anti-IL2R, | GB92 | >153 | Ongoing |
| Solumedrol and MMF) | GB27 | 92 | Rejection |
| Belatacept + | RUg13 | 306 | Planned Sac |
| Conventional | RTf14 | 287 | Rejection |
| Therapy (anti-IL2R, | RSl13 | 197 | Rejection |
| Solumedrol and MMF) | FA49 | 196 | Rejection |
| | RFi13 | 183 | Rejection |
| | Roe13 | 53 | Rejection |
| | RCz13 | 35 | Planned Sac |
| | RTi14 | 34 | Planned Sac |
| | RPb13 | 29 | Rejection |
| | RYg14 | 7 | Rejection |

TABLE 20

| | Recipient | Graft Survival | |
|---|---|---|---|
| Therapy | ID | (days | Comments |
| Anti-CD28 dAb | DX5R | >63 | Ongoing |
| (20 mg/kg) + | DX9X | >63 | Ongoing |
| Anti-CD154 dAb | | | |
| Anti-CD154 dAb | DX6L | >190 | Ongoing |
| (30 mg/kg) + | RHm14 | >188 | Ongoing |
| Conventional | GB94 | >175 | Ongoing |
| Therapy | GB34 | >162 | Ongoing |
| | RGm14 | 119 | Rejection |

For the combined anti-CD154 dAb and conventional therapy, the anti-CD154 dAb was administered 30 mg/kg intravenously each week from POD 0 to POD 70, then the anti-CD 154 dAb was administered biweekly from POD 70 to POD 140 without conventional therapy, and then the anti-CD154 dAb was administered at 30 mg/kg intravenously monthly after POD 140.

The anti-CD28 dAb is referred to herein as BMS-931699, which is a PEGylated anti-CD28 dAb as described in co-assigned U.S. Pat. No. 8,168,759. The PEG moiety is a 40 kDa branched polyethylene glycol. The sequence of a anti-CD28 dAb is as follows:

(SEQ ID NO: 26)
DIQMTQSPSSLSASVGDRVTITCRASRPIWPFLEWYQQKPGKAPKWYFTS
RLRHGVPSRFSGSGSGTCFTLTISSLOPEDFATYYCLQNVANPATFSQGT
KVEIKR

Although the embodiments have been described in detail with reference to examples above, it is understood that various modifications can be made without departing from the spirit of these embodiments, and would be readily known to the skilled artisan.

What is claimed is:

1: A method of treating renal transplant rejection comprising administering a therapeutically effective amount of BMS2h-572-633-CT-L2 (SEQ ID NO: 1) to a patient in need thereof.

2: The method of claim 1, wherein the transplant rejection is an acute transplant rejection.

3: The method of claim 1, wherein the transplant rejection is a chronic transplant rejection.

4: The method of claim 1, comprising administering a BMS2h-572-633-CT-L2 (SEQ ID NO: 1) dose from about 2 to about 30 mg/kg patient weight.

5: The method of claim 1, comprising administering a BMS2h-572-633-CT-L2 (SEQ ID NO: 1) dose from about 20 to about 30 mg/kg patient weight.

6: The method of claim 1, comprising administering a BMS2h-572-633-CT-L2 (SEQ ID NO: 1) at dose of about 20 mg/kg patient weight.

7: The method of claim 1, wherein BMS2h-572-633-CT-L2 (SEQ ID NO: 1) is administered with an immunosuppressive/immunomodulatory and/or anti-inflammatory agent.

8: The method of claim 7, wherein said immunosuppressive/immunomodulatory and/or anti-inflammatory agent is a CTLA4 mutant molecule.

9: The method of claim 8, wherein the CTLA4 mutant molecule is L104EA29Y-Ig (Belatacept).

10: The method of claim 9, wherein L104EA29Y-Ig (Belatacept) is administered at a dose from about 10 mg/kg to about 20 mg/kg patient weight.

11: The method of claim 10, wherein L104EA29Y-Ig (Belatacept) is administered at a dose of about 20 mg/kg patient weight.

12: The method of claim 1, wherein BMS2h-572-633-CT-L2 (SEQ ID NO: 1) is administered on a weekly basis during the duration of the treatment regimen.

13: The method of claim 7, wherein said immunosuppressive, immunomodulatory and/or anti-inflammatory agent is administered together with BMS2h-572-633-CT-L2 (SEQ ID NO: 1) on a weekly basis during the duration of the treatment regimen.

14: The method of claim 12, wherein the duration of the treatment regimen is 70 days.

15: The method of claim 1, wherein BMS2h-572-633-CT-L2 (SEQ ID NO: 1) is administered intravenously.

16: The method of claim 7, wherein said immunosuppressive, immunomodulatory and/or anti-inflammatory agent is administered intravenously.

17: The method of claim 1, wherein said patient further receives a conventional therapy for treatment of renal transplant rejection.

18: The method of claim 17, wherein the conventional therapy is a combination of an anti-IL-2R antibody, solumedrol, and mycophenolate mofetil (MMF).

19: The method of claim 1, wherein said immunosuppressive/immunomodulatory and/or anti-inflammatory agent is an anti-CD28 dAb.

20: The method of claim 19, wherein said anti-CD28 dAb comprises SEQ ID NO: 26.

21: The method of claim 20, wherein the anti-CD28 dAb is pegylated.

22: The method of claim 21, wherein the pegylated anti-CD28 dAb is pegylated with a 40 kD branched polyethylene glycol.

23: The method of claim 20, wherein the anti-CD28 dAb is Administered at a dose of about 1 mg/kg to about 10 mg/kg patient weight.

24: The method of claim 23, wherein the anti-CD28 dAb is administered at a dose of about 3 mg/kg patient weight at weekly intervals.
